(19) United States
(12) Patent Application Publication
(10) Pub. No.: US 2025/0288335 A1
(43) Pub. Date: Sep. 18, 2025

(54) HUMERUS AND ULNA STABILIZATION SYSTEMS

(71) Applicant: GLOBUS MEDICAL, INC., AUDUBON, PA (US)

(72) Inventors: Eric Obenschain, Elverson, PA (US); Mark Moutinho, Philadelphia, PA (US)

(21) Appl. No.: 18/607,837

(22) Filed: Mar. 18, 2024

(51) Int. Cl. A61B 17/80

(52) U.S. Cl. CPC A61B 17/8061; A61B 17/8023; A61B 17/8052

(57) ABSTRACT

A plate configured to support a bone extends longitudinally to define a proximal end and a distal end opposite the proximal end. The plate includes a first surface and second surface opposite the first surface. At least a portion of the first surface is configured to contact a surface of the bone. The plate includes openings spaced apart from one another along the plate. Each of the openings defines a fastener insertion axis for a fastener to be inserted therethrough. The plate includes apertures that are each adjacent to at least one of the openings. Each of the apertures defines a stabilizer insertion axis for a stabilizer to be inserted therethrough. The first surface of the plate is shaped to contour to a portion of the surface of the bone that extends along at least one of a. epiphysis, a metaphysis, or a diaphysis of the bone.

### TECHNICAL FIELD

The present disclosure relates to stabilization systems for trauma applications. More particularly, the present disclosure relates to stabilization systems for distal humerus fractures and proximal ulna fractures.

### BACKGROUND

Bone fractures are often repaired by internal fixation of the bone using one or more plates. The plate is held against the fractured bone with screws, for example, which engage the bone. The plate and bone are thus forced against each other in a manner that transfers load primarily between a bone contacting surface of the plate and the bone surface to reinforce the fractured bone during healing. This manner of plating generally creates relatively low stress concentration in the bone, as there may be a large contact area between the plate and the bone surface permitting transfer of load to be dispersed. There may be a desire to use locking screws, non-locking screw, or a combination of both that are able to dynamically compress the bone. Of course, the designs of the plates, types of screws, and locking and/or non-locking capabilities may vary based on the location and type of fracture.

Three long bones of the upper extremity are the humerus, radius, and ulna. In the case of distal humeral fracture fixation, plating of the lateral bone surface, the posterolateral bone surface, and/or the medial bone surface of the distal humerus may be desired. In the case of proximal ulnar fracture fixation, plating of the posterior bone surface of the proximal ulna may be desired. The geometry of these surfaces often requires intra-operative bending of an applicable plate to contour the plate to the bone surface. Thus, there remains a need for improved plating systems that provide appropriate stabilization of the distal humerus and proximal ulna.

### SUMMARY

To meet this and other needs, devices, systems, and methods of bone stabilization are provided, for example, for humerus stabilization. The distal humerus stabilization systems may include one or more plates and one or more fasteners. Although generally described with reference to the humerus, it will be appreciated that the stabilization systems described herein may be used or adapted to be used for fixation of other long bones as well, such as the femur, tibia, etc.

One aspect of the present disclosure is a plate configured to support a humerus. The plate extends longitudinally to define a proximal end and a distal end opposite the proximal end. The plate includes a first surface and a second surface opposite the first surface. At least a portion of the first surface is configured to contact a surface of the humerus. The second surface is beveled at the proximal end of the plate to form a beveled portion. The plate further includes openings spaced apart from one another along the plate that each define a fastener insertion axis for a fastener to be inserted therethrough. The plate also includes aperture that are each adjacent to at last one of the openings. Each of the apertures define a stabilizer insertion axis for a stabilizer to be inserted therethrough. The stabilizer insertion axis is generally parallel with the fastener insertion axis of the at last one of the openings to which each of the apertures is adjacent. At least one of the apertures extends through the beveled portion. The first surface of the plate is shaped to contour to a portion of the surface of the humerus that extends along at last one of a distal epiphysis, a distal metaphysis, or a diaphysis of the humerus.

Another aspect of the present disclosure is a plate configured to support a humerus. The plate extends longitudinally to define a proximal end and a distal end opposite the proximal end. The plate includes a first surface and a second surface opposite the first surface. At least a portion of the first surface is configured to contact a surface of the humerus. The plate further includes openings spaced apart from one another along the plate. Each of the openings defines a nominal fastener insertion axis and a conical range of fastener insertion axes surrounding the nominal fastener insertion axis for fasteners to be inserted therethrough. At least some of the openings include locking feature configured to prevent respective ones of the fasteners from loosening after being inserted therethrough. At least some of the openings are staggered about a center line that substantially longitudinally bisects the plate. The plate also includes apertures that are each adjacent to at least one of the openings. Each of the apertures defines a stabilizer insertion axis for a stabilizer to be inserted therethrough. The first surface of the plate is shaped to contour to a portion of the surface of the humerus that extends from at least one of a lateral epicondyle, a medial epicondyle, or a trochlea of the humerus to a location along the humerus that is proximal relative to the lateral epicondyle, the medial epicondyle, and the trochlea of the humerus.

Yet another aspect of the present disclosure is a system for stabilizing a humerus that includes a first plate and a second plate that each extend longitudinally to define a proximal end, a distal end opposite the proximal end, and a length extending therebetween. The first plate and the second plate each include a first surface and a second surface opposite the first surface. At least a portion of the first surface is configured to contact a surface of the humerus. The first plate and the second plate each also include openings spaced apart from one another along the length of each respective plate. Each of the openings defines a nominal fastener insertion axis and a conical range of fastener insertion axes surrounding the nominal fastener insertion axis for respective fasteners to be inserted therethrough. The first surface of the first plate is shaped to contour to a first portion of the surface of the humerus that includes a medial epicondyle of the humerus. The first surface of the second plate is shaped to contour to a second portion of the surface of the humerus that includes a lateral epicondyle of the humerus. Each of the openings of the first plate and the second plate defines the nominal fastener insertion axis to not intersect with the nominal fastener insertion axis of any other of the openings of the first plate and the second plate.

Yet another aspect of the present disclosure is a plate configured to support an ulna. The plate extends longitudinally to define a proximal end and a distal end opposite the proximal end. The plate includes a first surface and a second surface that is opposite the first surface. At least a portion of the first surface is configured to contact a surface of the ulna. The plate also includes first openings spaced apart from one another along a shaft portion of the plate that corresponds to at last one of a proximal metaphysis or a diaphysis of the ulna. The plate also includes second openings spaced apart from one another along a proximal portion of the plate that corresponds to at least one of the proximal metaphysis or a proximal epiphysis of the ulna. The first openings and the second openings each define a fastener insertion axis for a fastener to be inserted therethrough. At least some of the second openings define the fastener insertion axis to be generally normal to the second surface of the plate. A diameter of each of the first openings is larger than a diameter of each of the second openings. The plate also includes aperture that are each adjacent to at least one of the first openings or the second openings. Each of the apertures defines a stabilizer insertion axis for a stabilizer to be inserted therethrough. The stabilizer insertion axis may be generally parallel with the fastener insertion axis of the at least one of the first openings or the second opening to which each of the apertures is adjacent. The first surface of the plate is shaped to contour to a portion of the surface of the ulna that extends along an olecranon of the ulna.

Yet another aspect of the present disclosure is a plate configured to support an ulna. The plate extends longitudinally to define a proximal end and a distal end that is opposite the proximal end. The plate includes a first surface and a second surface opposite the first surface. At least a portion of the first surface is configured to contact a surface of the ulna. The plate also includes openings spaced apart from one another along the plate. At least some of the openings define a nominal fastener insertion axis and a conical range of fastener insertion axes surrounding the nominal fastener insertion axis for a fastener to be inserted therethrough. The plate also includes apertures each adjacent to at least one of the openings. Each of the apertures defines a stabilizer insertion axis for a stabilizer to be inserted therethrough. The stabilizer insertion axis is generally parallel with the nominal fastener insertion axis of the at least one of the openings to which each of the apertures is adjacent. The first surface of the plate is shaped to contour to a portion of the surface of the ulna that extends along an olecranon of the ulna.

Yet another aspect of the present disclosure is a plate configured to support an ulna. The plate extends longitudinally to define a proximal end and distal end that is opposite the proximal end. The plate includes a proximal portion located at the proximal end of the plate and a shaft portion located at the distal end of the plate. Each of the proximal portion and the shaft portion include a first surface and a second surface that is opposite the first surface. At least a portion of the first surface is configured to contact a surface of the ulna. Each of the proximal portion and the shaft portion also include at least one opening that defines a fastener insertion axis for a fastener to be inserted therethrough. Each of the proximal portion and the shaft portion also include at least one aperture that defines a stabilizer insertion axis for a stabilizer to be inserted therethrough. The plate also includes a band that connects the proximal portion to the shaft portion. The band is curved to contour around a posterior surface of an olecranon of the ulna. The at least one opening of the proximal portion defines the fastener insertion axis to aim distally toward a diaphysis of the ulna. The at least one opening of the shaft portion defines the fastener insertion axis to target a coronoid of the ulna.

### BRIEF DESCRIPTION OF THE DRAWINGS

A more complete understanding of the present invention, and the attendant advantages and features thereof, will be more readily understood by reference to the following detailed description when considered in conjunction with the accompanying drawings.

FIG. 1 is a side view of a first example of a distal humerus stabilization system including a first example of a medial distal humerus plate and a first example of a posterolateral distal humerus plate distal humerus plate cooperatively supporting a humerus.

FIG. 2A is a perspective view of the medial distal humerus plate of FIG. 1.

FIG. 2B is a side view of the medial distal humerus plate of FIG. 1.

FIG. 3 is a perspective view of a second example of a medial distal humerus plate.

FIG. 4 is a side view of a third example of a medial distal humerus plate supporting a humerus.

FIG. 5A is a perspective view of the medial distal humerus plate of FIG. 4.

FIG. 5B is a side view of the medial distal humerus plate of FIG. 4.

FIG. 6A is a perspective view of the posterolateral distal humerus plate of FIG. 1.

FIG. 6B is a side view of the posterolateral distal humerus plate of FIG. 1.

FIG. 7 is a perspective view of a second example of a posterolateral distal humerus plate.

FIG. 8 is a side view of a second example of a distal humerus stabilization system including the medial distal humerus plate of FIG. 1, and a first example of a lateral distal humerus plate distal humerus plate cooperatively supporting a humerus.

FIG. 9A is a perspective view of the lateral distal humerus plate of FIG. 8.

FIG. 9B is a side view of the lateral distal humerus plate of FIG. 8.

FIG. 10 is a perspective view of a second example of a lateral distal humerus plate.

FIG. 11 is a perspective view of an example of an extra-articular distal humerus plate.

FIG. 12A is a side view of a first example of a proximal ulna plate supporting an ulna.

FIG. 12B is a rear view of the proximal ulna plate of FIG. 12A supporting an ulna.

FIG. 13A is a front view of the proximal ulna plate of FIG. 12A.

FIG. 13B is a top view of the proximal ulna plate of FIG. 12A.

FIG. 14A is a side view of a second example of a proximal ulna plate supporting an ulna.

FIG. 14B is a rear view of the proximal ulna plate of FIG. 14A supporting an ulna.

FIG. 15A is a front view of the proximal ulna plate of FIG. 14A.

FIG. 15B is a top view of the proximal ulna plate of FIG. 14A.

FIG. 16 is a side view of a third example of a proximal ulna plate supporting an ulna.

FIG. 17A is a perspective view of the proximal ulna plate of FIG. 16.

FIG. 17B is a top view of the proximal ulna plate of FIG. 16.

FIG. 18A is a side view of a fourth example of a proximal ulna plate supporting an ulna.

FIG. 18B is a front view of the proximal ulna plate of FIG. 18A supporting an ulna.

FIG. 19 is a perspective view of the proximal ulna plate of FIG. 18A.

FIG. 20 is a perspective view of a fifth example of a proximal ulna plate.

### DETAILED DESCRIPTION

Embodiments of the disclosure are generally directed to devices, systems, and methods for bone stabilization, especially distal humeral stabilization and proximal ulnar stabilization. Specifically, embodiments directed to distal humeral stabilization systems include plates configured to sit on a lateral surface, posterolateral surface, and/or a medial surface of the distal humerus to support fractured portions of the distal epiphysis, distal metaphysis, and/or diaphysis of the humerus. Furthermore, embodiments directed to proximal ulnar stabilization systems include plates configured to sit on a posterior surface of the proximal ulna to support fractured potions of the proximal epiphysis, proximal metaphysis, and/or diaphysis of the ulna.

The plates described herein may be comprised of titanium, stainless steel, cobalt chrome, carbon composite, plastic or polymer-such as polyetheretherketone (PEEK), polyethylene, ultra-high molecular weight polyethylene (UHMWPE), resorbable polylactic acid (PLA), polyglycolic acid (PGA), combinations or alloys of such materials or any other appropriate material that has sufficient strength to be secured to and hold bone, while also having sufficient biocompatibility to be implanted into a body. Similarly, the fasteners described herein may be comprised of titanium, cobalt chrome, cobalt-chrome-molybdenum, stainless steel, tungsten carbide, combinations or alloys of such materials or other appropriate biocompatible materials. Although the above list of materials includes many typical materials of which plates and fasteners are made, it should be understood that the plates and fasteners of any appropriate material are contemplated.

The embodiments of the disclosure and the various features and advantageous details thereof are explained more fully with reference to the non-limiting embodiments and examples that are described and/or illustrated in the accompanying drawings and detailed in the following description. The features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiment of the disclosure. Accordingly, the examples and embodiments herein should not be construed as limiting the scope of the disclosure, which is defined solely by the appended claims and applicable law. Moreover, it is noted that lie reference numerals represent similar features and structures throughout the several views of the drawings.

### Distal Humerus Stabilization System

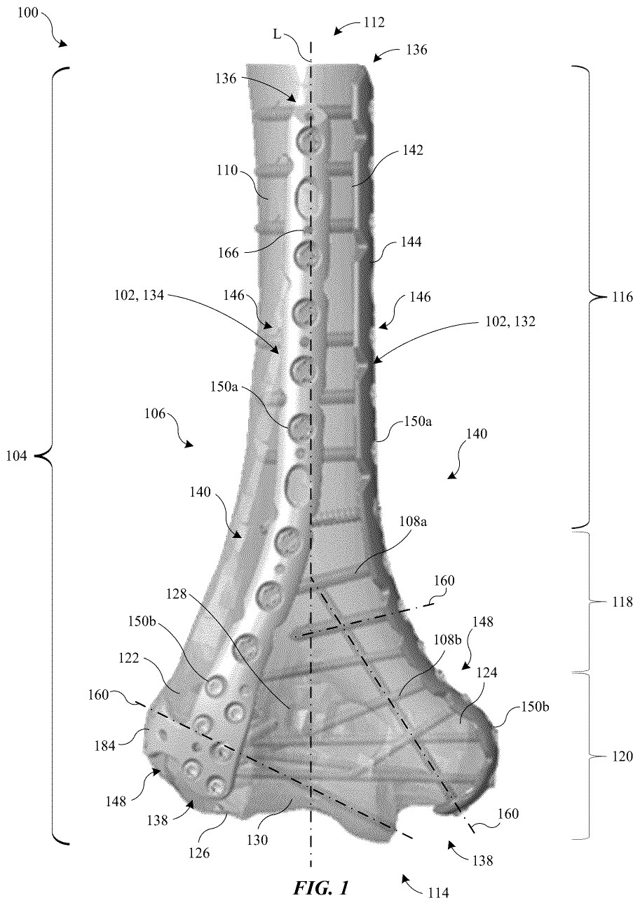

Referring now to the drawings, FIG. 1 shows a distal humerus stabilization system 100 according to a first example that utilizes two distal humerus plates 102 to cooperatively stabilize a distal aspect 104 of a humerus 106. Implementations of the distal humerus plates 102 described herein are configured for internal fixation of the humerus 106, in which the distal humerus plates 102 are secured directly to the humerus 106 via fasteners 108a and 108b collectively referred to as fasteners 108 (e.g., bone screws) such that the distal humerus plates 102 sit on (e.g., contact) a surface 110 of the humerus 106 while being positioned beneath adjacent soft tissue. By securing the distal humerus plates 102 directly to the surface 110 of the humerus 106, load may be transferred from the humerus 106 to the distal humerus plates 102 to support the humerus 106 in a fixed position while maintaining fracture reduction during healing. Internal fixation may be utilized to treat a variety of injuries and conditions where controlled (e.g., supported) healing of bone may be desired. With respect to internal fixation of the distal aspect 104 of the humerus 106, some such injuries may include intra-articular or extra-articular distal humerus fractures, nonunion or malunion repairs, distal humerus fractures that include osteopenic bone, commuted distal humerus fractures, and multi-fragmentary distal humerus fractures. Although a handful of applicable injuries are described herein, other injuries and conditions benefitted by controlled healing of the distal aspect 104 of the humerus 106 are also contemplated by this disclosure.

The humerus 106 is a long bone in the arm or forelimb that runs from the shoulder to the elbow. Long bones may be generally described with respect to a proximal end 112 and a distal end 114 thereof, as well as with respect to regions of the long bone segmented along a longitudinal axis L. Beginning from the proximal end 112, the regions include a proximal epiphysis, a proximal metaphysis, a diaphysis 116, a distal metaphysis 118, and a distal epiphysis 120. Accordingly, a proximal aspect of the humerus 106—located toward the shoulder—may include the proximal epiphysis (not shown), the proximal metaphysis (not shown), and a portion of the diaphysis 116. Furthermore, the distal aspect 104 of the humerus 106—located toward the elbow—may include the distal epiphysis 120, the distal metaphysis 118, and another portion of the diaphysis 116. Additionally, the distal epiphysis 120 of the humerus 106 may be described with respect to regions thereof, including a lateral epicondyle 122, a medial epicondyle 124, a capitellum 126, an olecranon fossa 128, and a trochlea 130. Although generally described with reference to the humerus 106, it will be appreciated that the distal humerus stabilization system 100 described herein may be used or adapted to be used for internal fixation of other long bones as well, such as the femur, radius, tibia, etc. Furthermore, although the humerus 106 is generally depicted as a human humerus bone, it will be appreciated that the distal humerus stabilization system 100 described herein may also be used or adapted to be used for internal fixation of long bones of other mammals as well, such as dogs, cats, horses, etc.

The distal humerus stabilization system 100 shown in FIG. 1 includes a first and a second of the distal humerus plates 102 that are each configured to sit on different locations of the distal aspect 104 of the humerus 106. The first of the distal humerus plates 102, for example, is configured to sit on a medial side of the distal aspect 104. Accordingly, the first of the distal humerus plates 102 may be referred to as a medial distal humerus plate 132. The second of the distal humerus plates 102 is configured to sit on a posterolateral and/or lateral side of the distal aspect 104. Accordingly, the second of the distal humerus plates 102 may be referred to as a posterolateral distal humerus plate 134. Although many of the features of the distal humerus plates 102 are described with reference to the medial distal humerus plate 132, it will be appreciated that other of the distal humerus plates 102 described herein may include features similar to those of the medial distal humerus plate 132, except where expressly indicated.

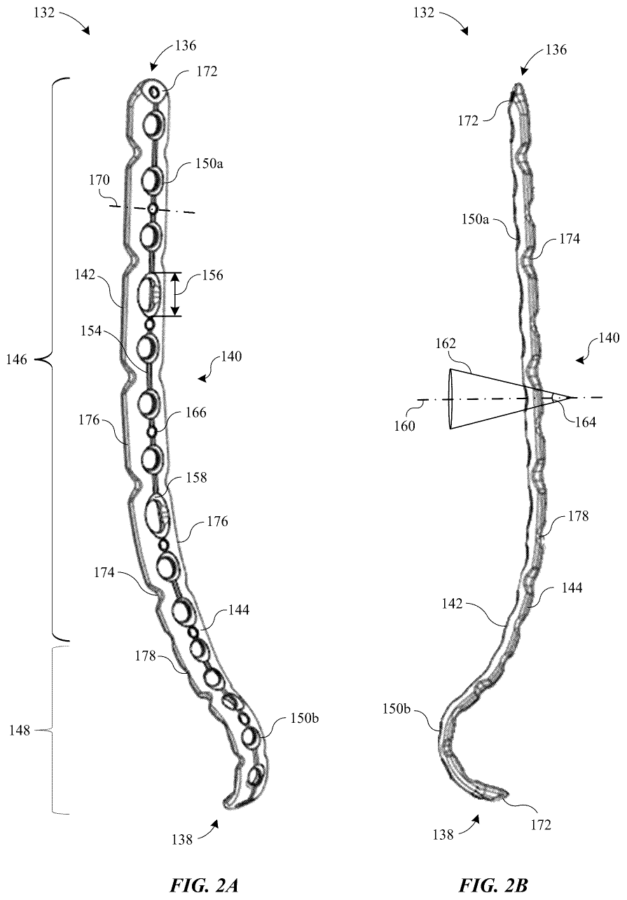

Referring now to FIGS. 1-2B, the medial distal humerus plate 132 extends longitudinally along the medial side of the distal aspect 104 to define a proximal end 136, a distal end 138, and a length 140 extending therebetween. The proximal end 136 may be configured to be positioned along the medial side of the diaphysis 116 of the humerus 106. The distal end 138 may be configured to be positioned along the medial side of the distal epiphysis 120 of the humerus 106 and may be configured to be positioned along the medial epicondyle 124 of the distal epiphysis 120 toward the trochlea 130. The medial distal humerus plate 132 includes a first surface 142 configured to contact the surface 110 of the humerus 106 and an opposite, second surface 144.

The medial distal humerus plate 132 may include a shaft portion 146 that is configured to extend along the diaphysis 116, the distal metaphysis 118, and/or portions thereof. The medial distal humerus plate 132 may also include a distal portion 148 that is configured to extend along the distal epiphysis 120, the distal metaphysis 118, and/or portions thereof. In other words, the shaft portion 146 may correspond to at least one of the diaphysis 116 or the distal metaphysis 118 of the humerus 106, and the distal portion 148 may correspond to at least one of the distal epiphysis 120 or the distal metaphysis 118 of the humerus 106.

Still referring to FIGS. 1-2B, the medial distal humerus plate 132 may include first openings 150a and second openings 150b, collectively referred to as openings 150 (e.g., sixteen of the openings 150) that are configured to receive the fasteners 108. The fasteners 108 may include locking fasteners, non-locking fasteners, or any other suitable fasteners known in the art. In some implementations, the fasteners 108 may include bone screws, such as fixed or variable angle bone screws. The fasteners 108 may also include other fasteners or features configured to secure to or engage with bone, such as nails, pikes, staples, pegs, barbs, hooks, or the like. The fasteners 108 may include a shaft portion configured to engage bone and a head portion configured to engage the medial distal humerus plate 132. Although the medial distal humerus plate 132 shown in FIGS. 1-2B comprises sixteen of the openings 150, other length implementations of the medial distal humerus plate 132 having different quantities and configurations of the openings 150 are also contemplated by this disclosure. For example, implementations of the medial distal humerus plate 132 may have lengths ranging from about 220 mm, in which the medial distal humerus plate 132 may include fourteen of the openings 150, to about 60 mm, in which the medial distal humerus plate 132 may include two of the openings

The openings 150 extend through the medial distal humerus plate 132 from the first surface 142 to the second surface 144. The openings 150 may be spaced apart from one another along the length 140 of the medial distal humerus plate 132. In some implementations, the openings 150 may be spaced apart from one another longitudinally along a center line 154 that longitudinally bisects the medial distal humerus plate 132 along the length 140. The medial distal humerus plate 132, however, may include any suitable number of the openings 150 in any suitable configuration. Surgeons may desire flexibility for placement of the fasteners 108 based on preference, anatomy, and fracture location. Furthermore, surgeons may have differing opinions as to the number, location, and types of the fasteners 108 used to treat a given injury. Accordingly, the openings 150 described herein may provide surgeons a variety of options for securing the medial distal humerus plate 132 to the humerus 106.

For example, it may be desired that the fasteners 108 be configured to lock to the medial distal humerus plate 132 after installation. Accordingly, one or more of the openings 150 may include locking features that interface with corresponding features of the fasteners 108 to form a locking engagement therebetween. For example, where the fasteners 108 include bone screws, the one or more of the openings 150 may include threads, texture (e.g., knurling), or the like which form a locking engagement with corresponding features of the fasteners 108 (e.g., corresponding threads, texture, or the like on the head portion of the fasteners 108) after being inserted through the openings 150 and tightened.

Variable positioning of the fasteners 108 relative to the medial distal humerus plate 132 may also be desired. Accordingly, one or more of the openings 150 may include an oblong profile that defines a length 156 along which the fasteners 108 may be positioned. By enabling variable positioning of the fasteners 108 along the length 156 of the oblong profile, the medial distal humerus plate 132 may be movable relative to the humerus 106 (or fragments thereof) after installation (e.g., partial installation) of the fasteners 108. In some implementations, the length 156 of the oblong profile may be about 6 mm. Furthermore, the oblong profile of the one or more openings 150 may include ramped surfaces 158 on each end thereof to support dynamic compression of the humerus 106, which may be desired to treat certain fracture types (e.g., transverse fractures, etc.). To support dynamic compression, some of the fasteners 108 may include a rounded surface on the head portion that is configured to slide against the ramped surfaces 158 of the openings 150 to dynamically compress regions of the humerus 106 along the length 140 of the medial distal humerus plate 132 upon tightening of the fasteners 108. It will be recognized by those skilled in the art that dynamic tensioning can also be achieved using the described configuration.

Additionally, it may be desired that the fasteners 108 target certain regions of the humerus 106, such as fragmented regions of the humerus 106. Furthermore, it may be desired that the fasteners 108 target regions of the humerus 106 that are comprised of high quality (e.g., dense and/or healthy) bone to achieve a high degree of bone purchase. It may also be desired that the fasteners 108 avoid certain regions of the humerus 106 or other surgical hardware. For example, it may be desired that the fasteners 108 avoid regions of the humerus 106 comprised of low quality (e.g., soft and/or compromised) bone. As another example, it may be desired that the fasteners 108 avoid other of the fasteners 108 that are associated with the medial distal humerus plate 132 or that are associated with other of the distal humerus plates 102 that may also be installed on the humerus 106. As yet another example, it may be desired that the fasteners 108 avoid other surgical hardware that may be installed in the humerus 106 such as orthopedic implants and the like.

Accordingly, the openings 150 may be configured to aid surgeons in targeting and/or avoiding regions of the humerus 106 by defining a fastener insertion axis 160 (e.g., a trajectory) for each of the fasteners 108 to be inserted therethrough and into the humerus 106. For example, one or more of the openings 150 may be substantially cylindrical such that the fastener insertion axis 160 aligns with an axis of the cylindrical opening. As described above, however, surgeons may desire flexibility for placement of the fasteners 108 based on several factors. Therefore, to provide such flexibility, one or more of the openings 150 may be substantially conical (e.g., frustoconical) such as to provide surgeons a conical range of fastener insertion axes 162 for the fasteners 108 to be inserted therethrough. In such an implementation, the fastener insertion axis 160 may be referred to as a nominal fastener insertion axis and the conical range of fastener insertion axes 162 may surround the nominal fastener insertion axis. In some implementations, the conical range of fastener insertion axes 162 may define a cone having an aperture angle 164. In some implementations, the aperture angle 164 of the conical range of fastener insertion axes 162 may be about 20-degrees, 40-degrees, 60-degrees, or some other angle.

In some implementations, at least some of the openings 150 located on the shaft portion 146 may each define the fastener insertion axis 160 to be substantially normal to the second surface 144 of the medial distal humerus plate 132 such that the fasteners 108 have a substantially transverse trajectory into the humerus 106. Some of the openings 150 located on the shaft portion 146, however, may define the fastener insertion axis 160 to aim proximally, distally, or at some other angle relative to the humerus 106. The openings 150 located on the distal portion 148 may each define the fastener insertion axis 160 to target the lateral epicondyle 122, the trochlea 130, and/or other regions of the humerus 106. For example, as shown in FIG. 1, the first-most distal of the openings 150 may define the fastener insertion axis 160 to aim proximally along the longitudinal axis L of the humerus 106 toward the diaphysis 116. The second and third-most distal of the openings 150 may each define the fastener insertion axis 160 to aim transversely toward the lateral epicondyle 122 of the humerus 106. The fifth-most distal of the openings 150 may define the fastener insertion axis 160 to aim distally toward the trochlea 130 of the humerus 106. Finally, the fifth and sixth-most distal of the openings 150 may each define the fastener insertion axis 160 to aim distally toward the lateral epicondyle 122 of the humerus 106.

Furthermore, to avoid collisions during installation of the fasteners 108, each of the openings 150 may define the fastener insertion axis 160 such as to not intersect the fastener insertion axis 160 of any other of the openings 150 of the medial distal humerus plate 132. For example, as shown in FIG. 1, the first-most distal of the openings 150 may define the fastener insertion axis 160 to aim proximally near the fastener insertion axis 160 of many other of the openings 150. The fasteners 108 that are inserted along such a trajectory are often referred to as columnar fasteners and may be used to reduce and provide compression to transverse fractures, highly comminuted and irregular fractures, intra-articular humerus fractures, and the like. Surgeons often express difficulty when maneuvering other fasteners past such columnar fasteners. Thus, the fastener insertion axis 160 of each of the openings 150 may be carefully defined such as to avoid such collisions.

The fastener insertion axis 160 of each of the openings 150 is also defined so as to avoid collisions with the fasteners 108 that secure other of the distal humerus plates 102 to the humerus 106, such as the posterolateral distal humerus plate 134 shown in FIG. 1. For example, each of the openings 150 located on the shaft portion 146 of the medial distal humerus plate 132 may be offset from the fastener insertion axis 160 of each of the openings 150 of the posterolateral distal humerus plate 134 along the longitudinal axis L of the humerus 106. As another example, each of the openings 150 located on the distal portion 148 of the medial distal humerus plate 132 may be positioned and angled such as to avoid the fastener insertion axis 160 of any of the openings 150 of the posterolateral distal humerus plate 134.

Still referring to FIGS. 1-2B, the fasteners 108 may comprise the first fasteners 108a and the second fasteners 108b, in which the first fasteners 108a have a larger diameter than the second fasteners 108b. Accordingly, the openings 150 may comprise the first openings 150a and the second openings 150b, in which the first openings 150a have a larger diameter than the second openings 150b. In some implementations, the first fasteners 108a include 3.5 mm bone screws, and the second fasteners 108b include 2.5 mm bone screws. To provide robust fixation of the medial distal humerus plate 132 to the humerus 106, the first fasteners 108a may be desired along the shaft portion 146 to secure the medial distal humerus plate 132 to the diaphysis 116 and/or the distal metaphysis 118 of the humerus 106. Accordingly, the first openings 150a (e.g., ten of the first openings 150a) configured to receive the first fasteners 108a may be positioned along the shaft portion 146. Some of the first openings 150a (e.g., two of the first openings 150a) may include an oblong profile as described above. In the implementation shown in FIGS. 1-2B, beginning at the proximal end 136 and moving distally, every fourth of the first openings 150a includes an oblong profile. In other implementations, however, others of the openings 150 may include an oblong profile. On the other hand, to target specific regions of the distal aspect 104 of the humerus 106, the second fasteners 108b may be desired along the distal portion 148 to target the lateral epicondyle 122 and/or the trochlea 130 of the humerus 106 from the medial epicondyle 124. Accordingly, the second openings 150b (e.g., six of the second openings 150b) configured to receive the second fasteners 108b may be positioned along the distal portion 148.

To aid surgeons in the installation of the medial distal humerus plate 132 to the humerus 106, the medial distal humerus plate 132 may include apertures 166 (e.g., seven of the apertures 166) that are configured to receive respective stabilizers 168. The apertures 166 may generally have a smaller diameter than the openings 150. The stabilizers 168 may be inserted through respective ones of the apertures 166 to provisionally fix the medial distal humerus plate 132 to the humerus 106 prior to installation of the fasteners 108. In some implementations, the stabilizers 168 may include a Kirschner wire (“K-wire”). In other implementations, the stabilizers 168 may include pins or the like. The apertures 166 extend through the medial distal humerus plate 132 from the first surface 142 to the second surface 144. At least some of the apertures 166 may be adjacent to one or more of the openings 150. Thus, in the implementation shown in FIGS. 2A-2B where the openings 150 are positioned along the center line 154, the apertures 166 may also be positioned along the center line 154. In some implementations, five of the apertures 166 may be positioned along the shaft portion 146, in which one of the apertures 166 is each positioned at the proximal end 136 of the medial distal humerus plate 132 and, moving distally, between every second adjacent pair of the first openings 150a thereafter. Two of the apertures 166 may be positioned along the distal portion 148, in which one of the apertures 166 is each positioned, starting from the distal end 138 and moving proximally, between every third adjacent pair of the second openings 150b. In other implementations, however, the apertures 166 may be positioned along the medial distal humerus plate 132 according to some other configuration.

The apertures 166 may define a stabilizer insertion axis 170 (e.g., a trajectory) for the stabilizers 168 to be inserted therethrough and into the humerus 106. In some implementations, the apertures 166 may define the stabilizer insertion axis 170 to be substantially parallel with the fastener insertion axis 160 of the one or more openings 150 to which a respective one of the apertures 166 is adjacent. By defining the stabilizer insertion axis 170 to be substantially parallel with the fastener insertion axis 160 of adjacent ones of the openings 150, surgeons may be able to acquire a sense of the actual trajectory of the fasteners 108 into the humerus 106 prior to installation of the fasteners 108 by provisional installation of the stabilizers 168.

Edges and other protrusions and irregularities of the medial distal humerus plate 132 may irritate adjacent soft tissue post-operatively, especially around regions of the humerus 106 that have minimal adjacent soft tissue such as the medial epicondyle 124. Therefore, to mitigate soft tissue irritation post-operatively, the head portion of the fasteners 208 may be configured to be received by the openings 250 such that the head portion sits flush or recessed with respect to the second surface 144 of the medial distal humerus plate 132. Additionally, the second surface 144 of the medial distal humerus plate 132 may be beveled at the distal end 138 to form a beveled portion 172 that is configured to be positioned on the medial epicondyle 124 of the humerus 106. In some implementations, a periphery (e.g., or portions thereof) of the beveled portion 172 may be radiused (e.g., may include a radius of about 0.5 mm). By softening the edge of the medial distal humerus plate 132 at the distal end 138, soft tissue irritation around the medial epicondyle 124 may be mitigated or prevented.

Furthermore, surgeons may choose to install the medial distal humerus plate 132 by inserting the medial distal humerus plate 132 through soft tissue of an arm from a distal location along the arm such the medial distal humerus plate 132 slides under the soft tissue as it travels proximally toward the proximal end 112 of the humerus 106. Edges of the medial distal humerus plate 132 may irritate soft tissue along the humerus 106 when inserting the medial distal humerus plate 132 in such a manner. Therefore, to mitigate soft tissue irritation intra-operatively during installation of the medial distal humerus plate 132, the second surface 144 of the medial distal humerus plate 132 may also be beveled at the proximal end 136 to form another of the beveled portion 172 that may smoothly slide underneath soft tissue during installation. The beveled portion 172 of the proximal end 136 may be configured to sit on the diaphysis 116 of the humerus 106 and may also mitigate post-operative irritation of adjacent soft tissue.

As previously mentioned, surgeons may desire to provisional fix the medial distal humerus plate 132 to the humerus 106 prior to installation of the fasteners 108. This may be particularly useful at the proximal end 136 of the medial distal humerus plate 132 while fitting the distal portion 148 to the distal aspect 104 of the humerus 106. Accordingly, one or more of the apertures 166 may be located on the beveled portion 172 of the proximal end 136 such that one of more of the stabilizers 168 may be inserted therethrough to provisionally fix the proximal end 136 of the medial distal humerus plate 132 to the diaphysis 116 of the humerus 106.

Due to the complex geometry of the distal aspect 104 of the humerus 106, especially with respect to the distal epiphysis 120 and the distal metaphysis 118, surgeons are often required to bend bone plates to some extent, pre-operatively and/or intra-operatively, to contour the bone plate to the distal aspect 104 of the humerus 106. Thus, to reduce the amount of bending required (or eliminate the need for bending altogether), the medial distal humerus plate 132 may include an anatomic contour configured to follow the best approximation of an average distal humerus anatomy. In particular, the first surface 142 of the medial distal humerus plate 132 may include an anatomic contour that substantially matches the surface 110 of the medial side of each of the medial epicondyle 124 of the distal epiphysis 120, the distal metaphysis 118, and/or a portion of the diaphysis 116 of an average humerus. Accordingly, the first surface 142 of the medial distal humerus plate 132 along the shaft portion 146 may include an anatomic contour that substantially matches the surface 110 of the medial side of each of the diaphysis 116, the distal metaphysis 118, and/or portions thereof. Furthermore, the first surface 142 of the medial distal humerus plate 132 along the distal portion 148 may include an anatomic contour that substantially matches the surface 110 of the medial side of each of the medial epicondyle 124 of the distal epiphysis 120, the distal metaphysis 118, and/or portions thereof.

As best shown in FIGS. 2A-2B, to reduce the time and effort required to bend the medial distal humerus plate 132, the medial distal humerus plate 132 may include recesses 174 (e.g., scallop cuts) spaced apart from one another along sides 176 of the medial distal humerus plate 132. In some implementations, the recesses 174 may be positioned between select ones of the openings 150. For example, the medial distal humerus plate 132 shown in FIGS. 1-2B includes one of the recesses 174 on each of the sides 176 between the first and second-most proximal of the first openings 150a, and, moving distally, between every second adjacent pair of the first openings 150a thereafter. The medial distal humerus plate 132 may also include the recesses 174 on each of the sides 176 between the second and third-most distal and the fourth and fifth-most distal of the second openings 150b. In other implementations, however, the recesses 174 may be positioned along the medial distal humerus plate 132 according to some other configuration.

The recesses 174 may be in the form of partially cylindrical valleys cut along the sides 176 of the medial distal humerus plate 132. However, other geometries may be used. By removing material from the sides 176 of the medial distal humerus plate 132 at select locations, the time and effort required to bend the medial distal humerus plate 132 may be reduced without substantially compromising the structural integrity thereof. In addition to reducing the effort required to bend the medial distal humerus plate 132, the recesses 174 may also discourage warping of the openings 150, the apertures 166, and/or other features of the medial distal humerus plate 132 by directing bending stresses to the recesses 174. The recesses 174 may also aid in preserving blood supply to the humerus 106 (e.g., a periosteum of the humerus 106), and thereby prevent osteonecrosis, by reducing the contact area between the first surface 142 of the medial distal humerus plate 132 and the surface 110 of the humerus 106.

Additionally, to further reduce the contact area between the first surface 142 and the surface 110 of the humerus 106, and thereby further preserve blood supply to the humerus 106, the medial distal humerus plate 132 may include undercuts 178 spaced apart from one another longitudinally along the first surface 142 of the medial distal humerus plate 132. In some implementations, the undercuts 178 may be positioned between select ones of the openings 150. For example, the medial distal humerus plate 132 shown in FIGS. 1-2B includes one of the undercuts 178 between the second and third-most proximal of the first openings 150a, and, moving distally, between every second adjacent pair of the first openings 150a thereafter. The medial distal humerus plate 132 may also include one of the undercuts 178 between the first and second-most distal of the second openings 150b, and, moving proximally, between every second adjacent pair of the second openings 150b thereafter. However, in other implementations, the undercuts 178 may be positioned along the medial distal humerus plate 132 according to some other configuration.

The undercuts 178 may be in the form of partially cylindrical valleys cut between the sides 176 of the medial distal humerus plate 132. However, other geometries may be used. Similar to the recesses 174, the undercuts 178 may reduce the effort required to bend the medial distal humerus plate 132, as well as discourage warping of the openings 150, the apertures 166, and/or other features of the medial distal humerus plate 132 when bending. In some implementations, the recesses 174 and the undercuts 178 may be spaced such that one of a pair of the recesses 174 or the one of the undercuts 178 is positioned between each adjacent pair of the openings 150.

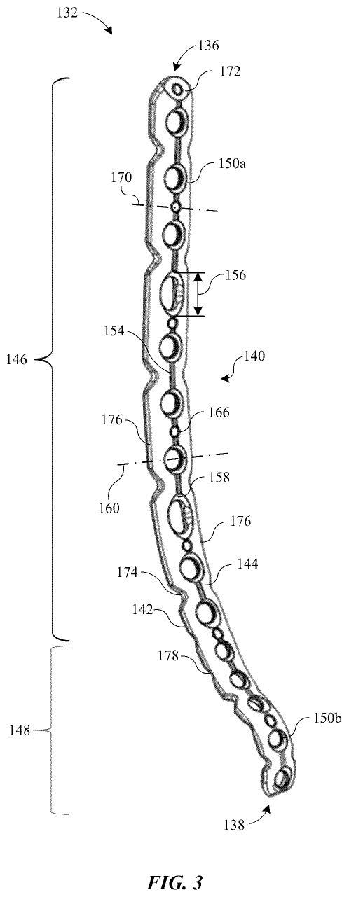

FIG. 3 shows another implementation of the medial distal humerus plate 132. As previously described above with reference to FIGS. 1-2B, the medial distal humerus plate 132 may be formed in a range of lengths. The medial distal humerus plate 132 shown in FIG. 3 is a shorter implementation of the medial distal humerus plate 132 of FIGS. 1-2B that omits the first-most distal of the second openings 150b. Accordingly, this implementation of the medial distal humerus plate 132 includes only fifteen of the openings 150, ten of which are the first openings 150a located on the shaft portion 146, and five of which are the second openings 150b located on the distal portion 148.

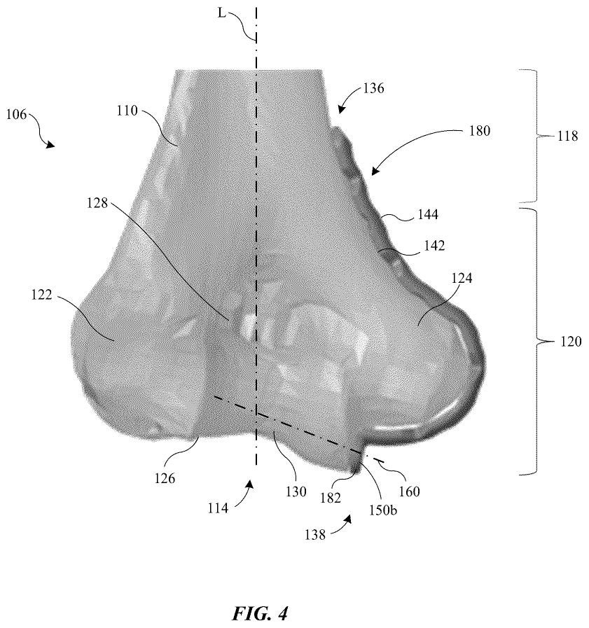

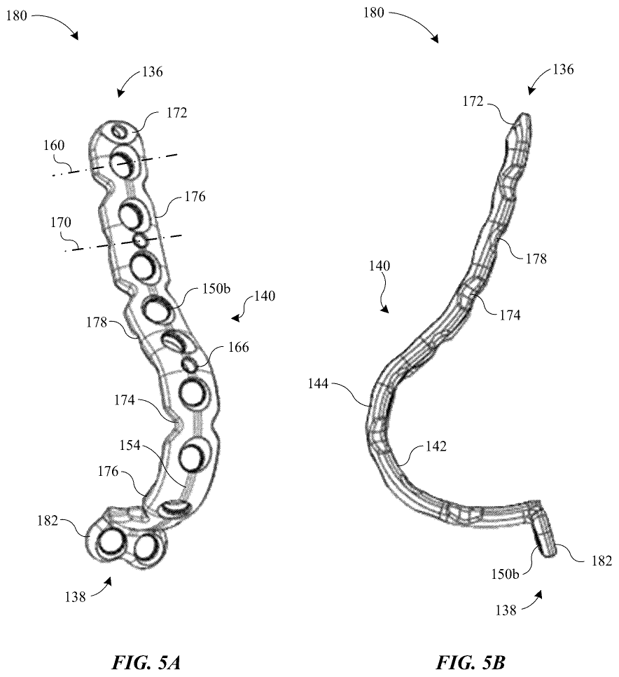

FIGS. 4-5B show another implementation of the medial distal humerus plate 132 that is shorter than those described above with reference to FIGS. 1-3. The medial distal humerus plate 132 shown in FIGS. 4-5B may be referred to as a medial shear distal humerus plate 180. The medial shear distal humerus plate 180 is substantially the same as the medial distal humerus plate 132 shown in FIGS. 1-2B except that the medial shear distal humerus plate 180 omits the shaft portion 146 thereof. Accordingly, the medial shear distal humerus plate 180 may include only the second openings 150b. By omitting the first openings 150a which are configured to receive the first fasteners 108a (e.g., the first fasteners 108a having a larger diameter than the second fasteners 108b), the medial shear distal humerus plate 180 may be capable of maintaining a lower profile (e.g., may be thinner) as compared to other implementations of the medial distal humerus plate 132. By maintaining a lower profile, soft tissue irritation may be mitigated. The second openings 150b may be positioned along the medial shear distal humerus plate 180 as described with respect to the second openings 150b of the medial distal humerus plate 132. Furthermore, each of the second openings 150b of the medial shear distal humerus plate 180 may each define the fastener insertion axis 160 as described with respect to the second openings 150b of the medial distal humerus plate 132.

The medial shear distal humerus plate 180 may also include a trochlear extension plate 182 that is configured to extend distally along a medial side of the trochlea 130. Accordingly, the first surface 142 of the medial shear distal humerus plate 180 may include an anatomical contour along the trochlear extension plate 182 that substantially matches the surface 110 of the medial side of the trochlea 130. The trochlear extension plate 182 of the medial shear distal humerus plate 180 may include two of the second openings 150b. In some implementations, the two of the second openings 150b of the trochlear extension plate 182 may be positioned laterally (e.g., horizontally) adjacent to one another. In such an implementation, the two of the second openings 150b of the trochlear extension plate 182 may each define the fastener insertion axis 160 to extend into the trochlea 130 from the medial side thereof. Accordingly, the trochlear extension plate 182 may enable reduction and support of highly distal regions of the humerus 106, which may be desired to treat certain injuries such as comminuted distal humerus fractures and fractures of highly distal regions of the humerus 106.

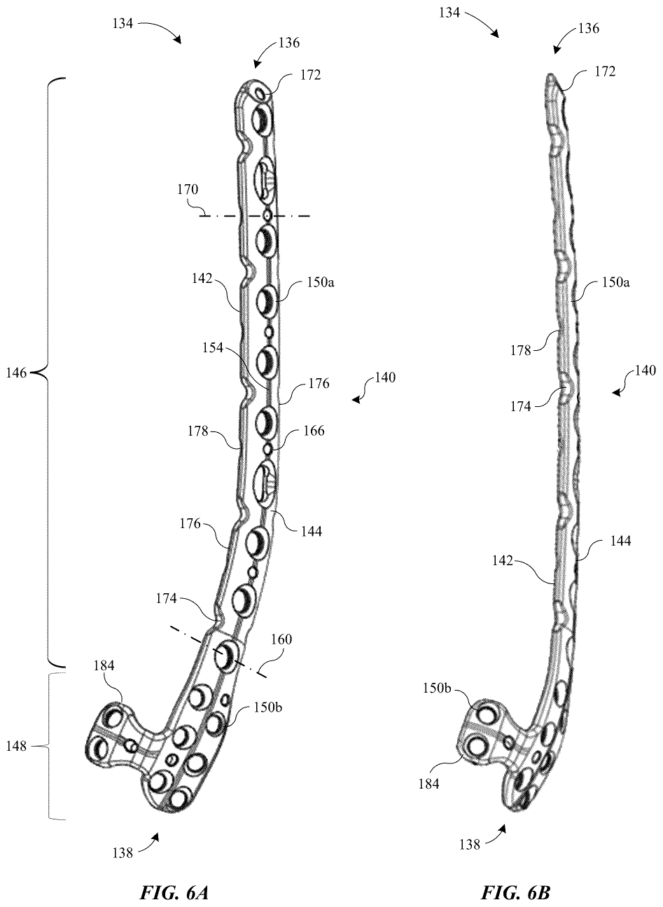

Referring now to FIGS. 1 and 6A-6B, the posterolateral distal humerus plate 134 extends longitudinally along the posterolateral and/or lateral side of the distal aspect 104. The posterolateral distal humerus plate 134 may be similar to the medial distal humerus plate 132 except as described herein. The proximal end 136 of the posterolateral distal humerus plate 134 may be configured to be positioned along the lateral side of the diaphysis 116 of the humerus 106. The distal end 138 of the posterolateral distal humerus plate 134 may be configured to be positioned along the posterolateral side of the distal epiphysis 120 of the humerus 106 and may be configured to be positioned along the posterolateral side of the lateral epicondyle 122.

The posterolateral distal humerus plate 134 shown in FIGS. 1 and 6A-6B comprises eighteen of the openings 150, including ten of the first openings 150a spaced apart from one another longitudinally along the shaft portion 146 of the posterolateral distal humerus plate 134 (e.g., along the center line 154), and six of the second openings 150b spaced apart from one another longitudinally along the distal portion 148 of the posterolateral distal humerus plate 134 in a staggered configuration (e.g., staggered about the center line 154). Staggering the second openings 150b may better enable targeted insertion of the fasteners 108 into the humerus 106. Furthermore, staggering the second openings 150b may also prevent the humerus 106 from splitting due to stresses associated with installation of the fasteners 108, which may be compounded if the fasteners 108 were to be installed linearly adjacent to one another.

Although the posterolateral distal humerus plate 134 shown in FIGS. 1 and 6A-6B comprises eighteen of the openings 150, longer and shorter length implementations of the posterolateral distal humerus plate 134 having different quantities and configurations of the openings 150 are also contemplated by this disclosure. For example, implementations of the posterolateral distal humerus plate 134 may have lengths ranging from about 286 mm, in which the posterolateral distal humerus plate 134 may include twenty-two of the openings 150, to about 51 mm, in which the posterolateral distal humerus plate 134 may include two of the openings 150.

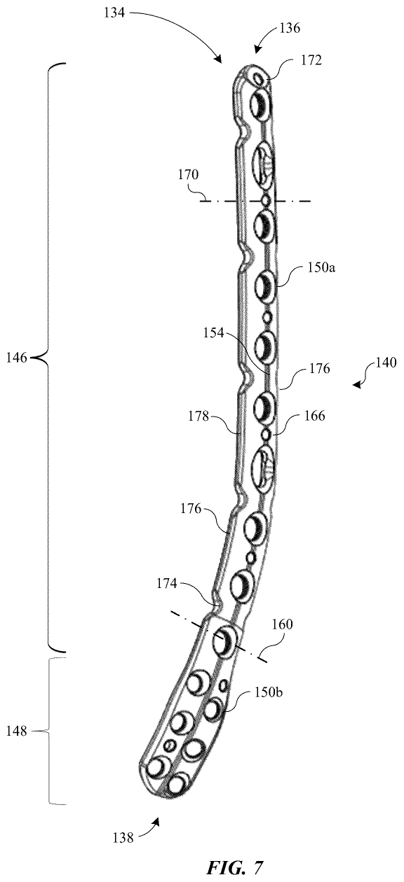

The posterolateral distal humerus plate 134 may include a lateral extension plate 184 that extends generally in lateral direction from the distal portion 148 to sit on a lateral side of the lateral epicondyle 122 of the humerus 106. Accordingly, the first surface 142 of the posterolateral distal humerus plate 134 along the lateral extension plate 184 may include an anatomical contour that substantially matches the surface 110 of a lateral side of the lateral epicondyle 122. The lateral extension plate 184 may include two of the second openings 150b. In some implementations, the two of the second openings 150b of the lateral extension plate 184 may be spaced longitudinally (e.g., vertically) adjacent to one another. The posterolateral distal humerus plate 134, however, may include any suitable number of the openings 150 in any suitable configuration. Referring now to FIG. 7, however, in some implementations the posterolateral distal humerus plate 134 may omit the lateral extension plate 184. In such an implementation, the posterolateral distal humerus plate 134 may include only sixteen of the openings 150, including ten of the first openings 150a and six of the second openings 150b.

Referring back to FIGS. 1 and 6A-6B, similar to the medial distal humerus plate 132, the posterolateral distal humerus plate 134 may be configured to support variable positioning of the fasteners 108 and dynamic compression of the humerus 106 (or portions thereof). Accordingly, in the implementation shown in FIGS. 1 and 6A-6B, beginning at the second-most proximal of the first openings 150a and moving distally, every fifth of the first openings 150a may include an oblong profile. In other implementations, however, others of the openings 150 may include an oblong profile.

The openings 150 of the posterolateral distal humerus plate 134 may each define the fastener insertion axis 160 (e.g., the nominal fastener insertion axis) to target certain regions of the humerus 106 while avoiding other regions and surgical hardware. Accordingly, in some implementations, at least some of the openings 150 located on the shaft portion 146 may define the fastener insertion axis 160 to be substantially normal to the second surface 144 of the posterolateral distal humerus plate 134 such that the fasteners 108 have a substantially transverse trajectory into the humerus 106. The openings 150 located on the distal portion 148 may each define the fastener insertion axis 160 to target the capitellum 126, the medial epicondyle 124, the trochlea 130 and/or other regions of the humerus 106 while avoiding regions such as the olecranon fossa 128. For example, as shown in FIG. 1, an upper of the two of the second openings 150b of the lateral extension plate 184 may define the fastener insertion axis 160 to aim distally toward the trochlea 130. A lower of the two of the second openings 150b of the lateral extension plate 184 may define the fastener insertion axis 160 to aim transversely toward to the medial epicondyle 124. Each of the six other of the second openings 150b of the distal portion 148 define the fastener insertion axis 160 to target the capitellum 126 or other regions of the humerus 106 while avoiding regions such as the olecranon fossa 128.

The fastener insertion axis 160 of each of the openings 150 of the posterolateral distal humerus plate 134 may also be defined so as to avoid collisions between the fasteners 108. Accordingly, each of the openings 150 may define the fastener insertion axis 160 such that it does not intersect the fastener insertion axis 160 of any other of the openings 150 of the posterolateral distal humerus plate 134. The fastener insertion axis 160 of each of the openings 150 is also defined so as to avoid collisions with the fasteners 108 that secure other of the distal humerus plates 102 to the humerus 106, such as the medial distal humerus plate 132 as shown in FIG. 1.

Still referring to FIGS. 1 and 6A-6B, to support provisional fixation of the posterolateral distal humerus plate 134 to the humerus 106, the posterolateral distal humerus plate 134 may include the apertures 166 (e.g., eight of the apertures 166) configured to receive respective ones of the stabilizers 168. The posterolateral distal humerus plate 134 shown in FIGS. 1 and 6A-6B includes five of the apertures 166 along the shaft portion 146, in which one of the apertures 166 is each positioned on the beveled portion 172 of the proximal end 136 and, moving distally, between every second adjacent pair of the first openings 150a thereafter. The posterolateral distal humerus plate 134 may also include three of the apertures 166 on the distal portion 148, in which one of the apertures 166 is positioned on the lateral extension plate 184. In other implementations, however, the apertures 166 may be positioned along the posterolateral distal humerus plate 134 according to some other configuration.

Furthermore, to mitigate edge-related post-operative irritation of adjacent soft tissue, the second surface 144 of the posterolateral distal humerus plate 134 may include the beveled portion 172 on the proximal end 136, in which the beveled portion 172 may be configured to sit on a lateral side of the diaphysis 116 of the humerus 106. The beveled portion 172 of the proximal end 136 may also mitigate intra-operative irritation of soft tissue during installation of the posterolateral distal humerus plate 134 by smoothly sliding underneath soft tissue where a surgeon chooses to insert the posterolateral distal humerus plate 134 proximally from a distal location of the arm.

To reduce the amount of bending required to fit the posterolateral distal humerus plate 134 to the distal aspect 104 of the humerus 106, the posterolateral distal humerus plate 134 may include an anatomic contour configured to follow the best approximation of average distal humerus anatomy. In particular, the first surface 142 of the posterolateral distal humerus plate 134 may include an anatomic contour that substantially matches the surface 110 of the posterolateral and/or lateral side of each of the lateral epicondyle 122 of the distal epiphysis 120, the distal metaphysis 118, and/or a portion of the diaphysis 116 of an average humerus. Accordingly, the first surface 142 of the posterolateral distal humerus plate 134 along the shaft portion 146 may include an anatomic contour that substantially matches the surface 110 of the posterolateral and/or lateral side of each of the diaphysis 116, the distal metaphysis 118, and/or portions thereof. Furthermore, the first surface 142 of the posterolateral distal humerus plate 134 along the distal portion 148 may include an anatomic contour that substantially matches the surface 110 of the posterolateral side of each of the lateral epicondyle 122 of the distal epiphysis 120, the distal metaphysis 118, and/or portions thereof.

Furthermore, to reduce the time and effort required to bend the posterolateral distal humerus plate 134 to fit the distal aspect 104 of the humerus 106, the posterolateral distal humerus plate 134 may include the recesses 174 spaced apart from one another along the sides 176 of the posterolateral distal humerus plate 134. For example, the posterolateral distal humerus plate 134 shown in FIGS. 1 and 6A-6B includes one of the recesses 174 on each of the sides 176 between the first and second-most proximal of the first openings 150a, and, moving distally, between every second adjacent pair of the first openings 150a thereafter. However, in other implementations, the recesses 174 may be positioned along the posterolateral distal humerus plate 134 according to some other configuration.

Additionally, to reduce the contact area between the first surface 142 and the surface 110 of the humerus 106, and thereby preserve blood supply to the humerus 106, the posterolateral distal humerus plate 134 may include the undercuts 178 spaced apart from one another along the first surface 142 of the posterolateral distal humerus plate 134. For example, the posterolateral distal humerus plate 134 shown in FIGS. 1 and 6A-6B includes one of the undercuts 178 between the second and third-most proximal of the first openings 150a, and, moving distally, between every second adjacent pair of the first openings 150a thereafter. However, in other implementations, the undercuts 178 may be positioned along the posterolateral distal humerus plate 134 according to some other configuration.

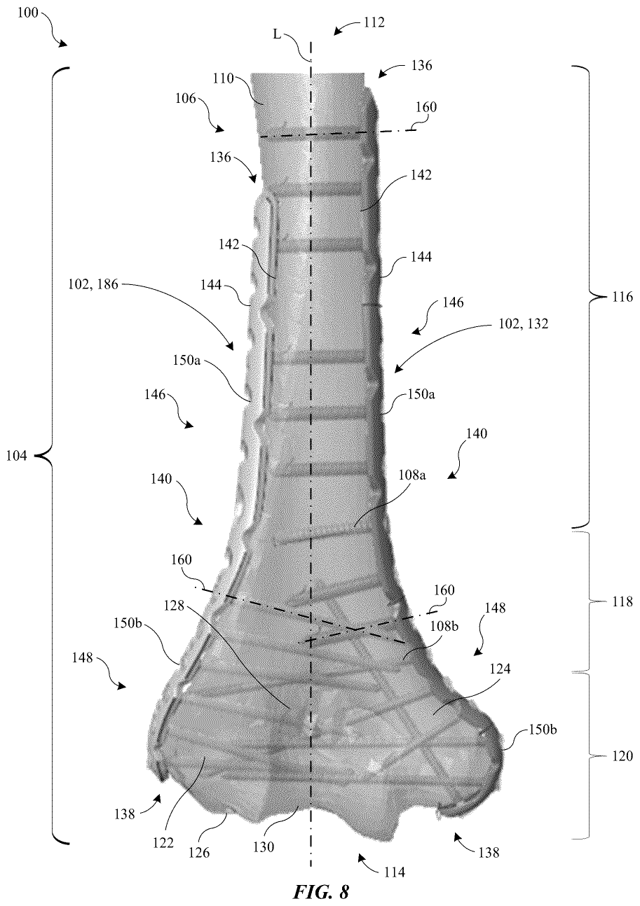

FIG. 8 shows the distal humerus stabilization system 100 according to a second example that utilizes two of the distal humerus plates 102 to cooperatively stabilize the distal aspect 104 of the humerus 106. The distal humerus stabilization system 100 shown in FIG. 8 includes the medial distal humerus plate 132 as described above with reference to FIGS. 1-2B, as well as a third of the distal humerus plates 102. The third of the distal humerus plates 102 is configured to sit on a lateral side of the distal aspect 104 of the humerus 106. Accordingly, the third of the distal humerus plates 102 may be referred to as a lateral distal humerus plate 186.

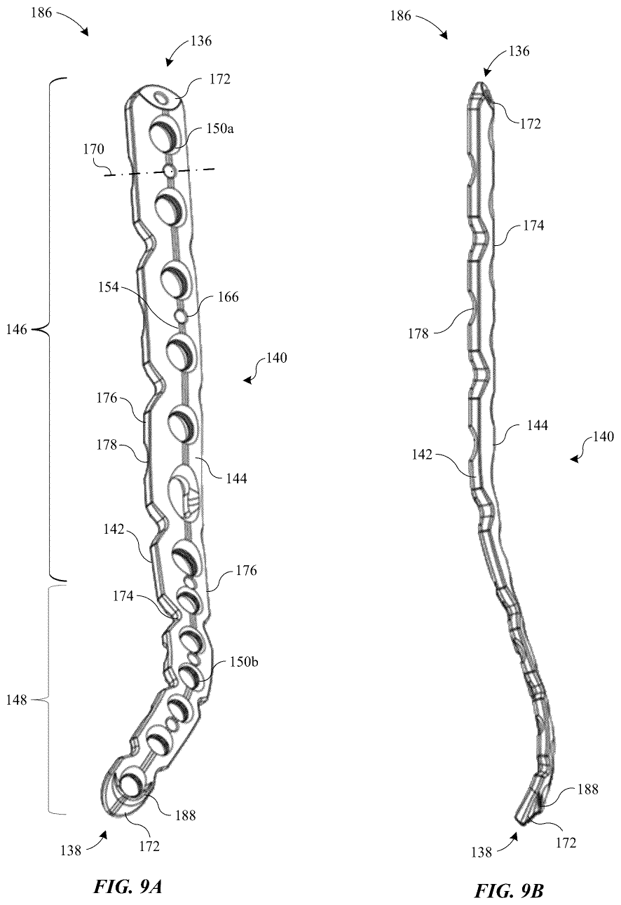

Referring now to FIGS. 8-9B, the lateral distal humerus plate 186 extends longitudinally along the lateral side of the distal aspect 104 of the humerus 106. The lateral distal humerus plate 186 may be similar to the medial distal humerus plate 132 except as described herein. The proximal end 136 of the lateral distal humerus plate 186 may be configured to be positioned along the lateral side of the diaphysis 116 of the humerus 106. The distal end 138 of the lateral distal humerus plate 186 may be configured to be positioned along the lateral side of the distal epiphysis 120 of the humerus 106 and may be configured to be positioned along the lateral side of the lateral epicondyle 122.

The lateral distal humerus plate 186 shown in FIGS. 8-9B comprises thirteen of the openings 150, including seven of the first openings 150a spaced apart from one another longitudinally along the shaft portion 146 of the lateral distal humerus plate 186 (e.g., along the center line 154), and six of the second openings 150b spaced apart from one another along the distal portion 148 of the lateral distal humerus plate 186 (e.g., along the center line 154). Although the lateral distal humerus plate 186 shown in FIGS. 8-9B comprises thirteen of the openings 150, longer and shorter implementations of the lateral distal humerus plate 186 having different quantities and configurations of the openings 150 are also contemplated. For example, implementations of the lateral distal humerus plate 186 may have lengths ranging from about 270 mm, in which the lateral distal humerus plate 186 may include nineteen of the openings 150, to about 66 mm, in which the lateral distal humerus plate 186 may include two of the openings 150.

Similar to the medial distal humerus plate 132, the lateral distal humerus plate 186 may be configured to support variable positioning of the fasteners 108 and dynamic compression of the humerus 106 (or portions thereof). Accordingly, in the implementation shown in FIGS. 8-9B, the sixth-most proximal of the first openings 150a includes an oblong profile. In other implementations, however, others of the openings 150 may include an oblong profile.

Also similar to the medial distal humerus plate 132, the openings 150 of the lateral distal humerus plate 186 may each define the fastener insertion axis 160 (e.g., the nominal fastener insertion axis) to target certain regions of the humerus 106 while avoiding other regions and surgical hardware. Accordingly, in some implementations, at least some of the openings 150 located on the shaft portion 146 may define the fastener insertion axis 160 to be substantially normal to the second surface 144 of the lateral distal humerus plate 186 such that the fasteners 108 have a substantially transverse trajectory into the humerus 106. The openings 150 located on the distal portion 148 may each define the fastener insertion axis 160 to target the medial epicondyle 124, the trochlea 130 and/or other regions of the humerus 106 while avoiding regions such as the olecranon fossa 128. For example, the first-most distal of the second openings 150b may define the fastener insertion axis 160 to aim transversely toward the trochlea 130 and/or the medial epicondyle 124. The remaining of the second openings 150b may each define the fastener insertion axis 160 to aim distally toward the medial epicondyle 124 and/or other regions of the distal aspect 104 of the humerus 106. In implementations where one or more of the openings 150 define the conical range of fastener insertion axes 162 to provide surgeons flexibility for insertion of the fasteners 108, the one or more of the openings 150 may include a protrusion 188 that extends around a portion of a circumference of the one or more of the openings 150. The protrusion 188 may extend the conical profile of the one or more of the openings 150 such that the aperture angle 164 of the conical range of fastener insertion axes 162 can be fully achieved. For example, in the implementation shown in FIGS. 8-9B, the first-most distal of the second openings 150b includes the protrusion 188 extending around a distal portion of a circumference of the first-most distal of the second openings 150b.

The fastener insertion axis 160 of the lateral distal humerus plate 186 may also be defined so as to avoid collisions between the fasteners 108. Accordingly, each of the openings 150 may define the fastener insertion axis 160 such that it does not intersect the fastener insertion axis 160 of any other of the openings 150 of the lateral distal humerus plate 186. The fastener insertion axis 160 of each of the openings 150 is also defined so as to avoid collisions with the fasteners 108 that secure other of the distal humerus plates 102 to the humerus 106, such as the medial distal humerus plate 132 as shown in FIG. 8. In some implementations, the fastener insertion axis 160 of each of the second openings 150b of the lateral distal humerus plate 186 may be defined to aim anteriorly relative to the fastener insertion axis 160 of each of the second openings 150b of the medial distal humerus plate 132.

As best shown in FIGS. 9A-9B, to support provisional fixation of the lateral distal humerus plate 186 to the humerus 106, the lateral distal humerus plate 186 may include the apertures 166 (e.g., six of the apertures 166) configured to receive respective ones of the stabilizers 168. The lateral distal humerus plate 186 shown in FIGS. 9A-9B includes two of the apertures 166 along the shaft portion 146, in which one of the apertures 166 is each positioned on the beveled portion 172 of the proximal end 136, between the first and second-most proximal of the openings 150, and between the third and fourth-most proximal of the openings 150. The lateral distal humerus plate 186 may include three of the apertures 166 on the distal portion 148, in which one of the apertures 166 is each positioned between the second and third-most distal of the openings 150, between fourth and fifth-most distal of the openings 150, and between the sixth and seventh-most distal of the openings 150. In other implementations, however, the apertures 166 may be positioned along the lateral distal humerus plate 186 according to some other configuration.

Furthermore, to mitigate edge-related post-operative irritation of adjacent soft tissue, the second surface 144 of the lateral distal humerus plate 186 may include the beveled portion 172 on each of the proximal end 136 and the distal end 138. The beveled portion 172 of the distal end 138 may be configured to sit on a lateral side of the lateral epicondyle 122 and the beveled portion 172 of the proximal end 136 may be configured to sit on a lateral side of the diaphysis 116 of the humerus 106. The beveled portion 172 of the proximal end 136 may also mitigate intra-operative irritation of soft tissue during installation of the lateral distal humerus plate 186 by smoothly sliding underneath soft tissue where a surgeon chooses to insert the lateral distal humerus plate 186 proximally from a distal location of the arm.

To reduce the amount of bending required to fit the lateral distal humerus plate 186 to the distal aspect 104 of the humerus 106, the lateral distal humerus plate 186 may include an anatomic contour configured to follow the best approximation of average distal humerus anatomy. In particular, the first surface 142 of the lateral distal humerus plate 186 may include an anatomic contour that substantially matches the surface 110 of the lateral side of each of the lateral epicondyle 122 of the distal epiphysis 120, the distal metaphysis 118, and/or a portion of the diaphysis 116 of an average humerus. Accordingly, the first surface 142 of the lateral distal humerus plate 186 along the shaft portion 146 may include an anatomic contour that substantially matches the surface 110 of the lateral side of each of the diaphysis 116, the distal metaphysis 118, and/or portions thereof. Furthermore, the first surface 142 of the lateral distal humerus plate 186 along the distal portion 148 may include an anatomic contour that substantially matches the surface 110 of the lateral side of each of the lateral epicondyle 122 of the distal epiphysis 120, the distal metaphysis 118, and/or portions thereof.

Furthermore, to reduce the time and effort required to bend the lateral distal humerus plate 186 to fit the distal aspect 104 of the humerus 106, the lateral distal humerus plate 186 may include the recesses 174 spaced apart from one another along the sides 176 of the lateral distal humerus plate 186. For example, the lateral distal humerus plate 186 shown in FIGS. 8-9B includes one of the recesses 174 on each of the sides 176 between the second and third-most proximal of the openings 150, and, moving distally, between every second adjacent pair of the openings 150 thereafter. However, in other implementations, the recesses 174 may be positioned along the lateral distal humerus plate 186 according to some other configuration.

Additionally, to reduce the contact area between the first surface 142 of the lateral distal humerus plate 186 and the surface 110 of the humerus 106, and thereby preserve blood supply to the humerus 106, the lateral distal humerus plate 186 may include the undercuts 178 spaced apart from one another along the first surface 142 of the lateral distal humerus plate 186. For example, the lateral distal humerus plate 186 shown in FIGS. 8-9B includes one of the undercuts 178 between the first and second-most proximal of the first openings 150a, and, moving distally, between every second adjacent pair of the openings 150 thereafter. The lateral distal humerus plate 186 may also include one of the undercuts 178 between the first and second-most distal of the second openings 150b, and, moving proximally, between every second adjacent pair of the second openings 150b thereafter. In other implementations, however, the undercuts 178 may be positioned along the lateral distal humerus plate 186 according to some other configuration.

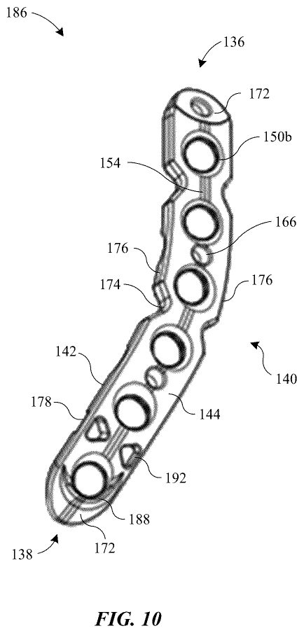

FIG. 10 shows another implementation of the lateral distal humerus plate 186 described above with reference to FIGS. 8-9B. As previously described, the lateral distal humerus plate 186 may be formed in a range of lengths. The lateral distal humerus plate 186 shown in FIG. 10 is a shorter implementation of the lateral distal humerus plate 186 of FIGS. 8-9B and may be referred to as a lateral shear distal humerus plate 190. The lateral shear distal humerus plate 190 is substantially the same as the lateral distal humerus plate 186 except that the lateral shear distal humerus plate 190 omits the shaft portion 146. Accordingly, the lateral shear distal humerus plate 190 may include only the second openings 150b, which may be positioned along the lateral shear distal humerus plate 190 as described with respect to the second openings 150b of the lateral distal humerus plate 186. Furthermore, the second openings 150b of the lateral shear distal humerus plate 190 each may define the fastener insertion axis 160 as described with respect to the second openings 150b of the lateral distal humerus plate 186.

The lateral shear distal humerus plate 190 may also include suture holes 192 (e.g., two of the suture holes 192) to enable provisional fixation of the lateral shear distal humerus plate 190 to adjacent soft tissue. The suture holes 192 may also facilitate repair of adjacent soft tissue in conjunction with installation of the lateral shear distal humerus plate 190. The suture holes 192 extend from the first surface 142 to the second surface 144 of the lateral shear distal humerus plate 190. In some implementations, the suture holes 192 may be substantially triangular, however, other geometries may be used. As shown in FIG. 10, the lateral shear distal humerus plate 190 may include two of the suture holes 192 positioned laterally (e.g., horizontally) adjacent to one another between the first and second-most distal of the second openings 150b. In other implementations, however, the suture holes 192 may be positioned at other locations along the lateral shear distal humerus plate 190.

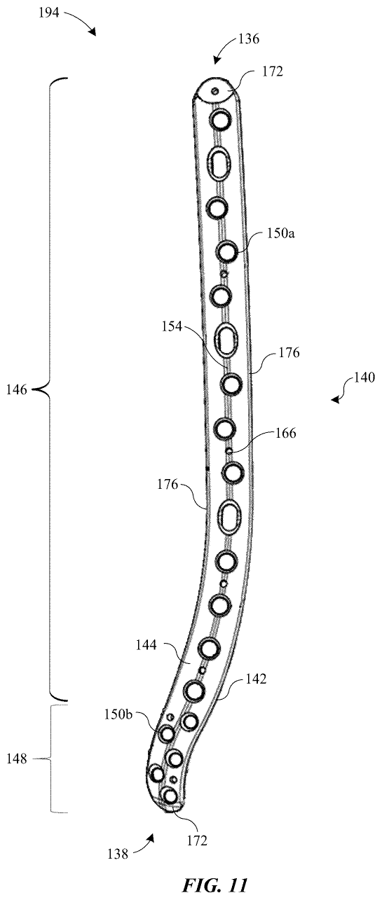

FIG. 11 shows a fourth of the distal humerus plates 102. The fourth of the distal humerus plates 102 is configured to sit on the posterolateral and/or lateral side of the humerus 106 and is configured primarily for the treatment of extra-articular distal humeral fractures along the diaphysis 116 and/or the distal metaphysis 118 of the humerus 106. Accordingly, the fourth of the distal humerus plates 102 may be referred to as an extra-articular distal humerus plate 194. The extra-articular distal humerus plate 194 may be similar to the medial distal humerus plate 132 except as described herein. In order to properly support the humerus 106 in the case of extra-articular fractures along the diaphysis 116 and/or the distal metaphysis 118, the extra-articular distal humerus plate 194 may be generally thicker and extend farther proximally toward the proximal end 112 of the humerus 106 than the previously described implementations of the distal humerus plates 102.

Although the extra-articular distal humerus plate 194 is configured primarily for the treatment of extra-articular distal humerus fractures, the extra-articular distal humerus plate 194 may also be used for the treatment of other injuries such as nonunion or malunion repairs, distal humerus fractures that include osteopenic bone, commuted distal humerus fractures, and multi-fragmentary distal humerus fractures. Although a handful of applicable injuries are described herein, other injuries and conditions benefitted by controlled healing of the distal aspect 104 of the humerus 106 are also contemplated by this disclosure.

The extra-articular distal humerus plate 194 extends longitudinally along the posterolateral and/or lateral side of the distal aspect 104. The proximal end 136 of the extra-articular distal humerus plate 194 may be configured to be positioned along the lateral side of the diaphysis 116 of the humerus 106. The distal end 138 of the lateral distal humerus plate 186 may be configured to be positioned along the posterolateral side of the distal epiphysis 120 of the humerus 106 and may be configured to be positioned along the posterolateral side of the lateral epicondyle 122.

The extra-articular distal humerus plate 194 shown in FIG. 11 comprises nineteen of the openings 150, including fourteen of the first openings 150a spaced apart from one another longitudinally along the shaft portion 146 of the extra-articular distal humerus plate 194 in a staggered configured (e.g., staggered about the center line 154), and five of the second openings 150b spaced apart from one another longitudinally along the distal portion 148 of the extra-articular distal humerus plate 194 in a staggered configuration (e.g., staggered about the center line 154). Staggering the first openings 150a may better enable the fastener insertion axis 160 of each of the first openings 150a to avoid surgical hardware that has been previously installed in the humerus 106. Such surgical hardware may include implants associated with a shoulder replacement or a reverse shoulder replacement or may include any other implants that extend into the diaphysis 116 of the humerus 106. Staggering the second openings 150b may better enable targeted insertion of the second fasteners 108b into the distal aspect 104 of the humerus 106. Furthermore, staggering the first openings 150a along the shaft portion 146 and the second openings 150b along the distal portion 148 may also prevent the humerus 106 from splitting due to the stresses associated with installation of the fasteners 108, which may be compounded if the fasteners 108 were to be installed linearly adjacent to one another.

Although the extra-articular distal humerus plate 194 shown in FIG. 11 comprises nineteen of the openings 150, longer and shorter implementations of the extra-articular distal humerus plate 194 having different quantities and configurations of the openings 150 are also contemplated. For example, implementations of the extra-articular distal humerus plate 194 may have lengths ranging from about 316 mm, in which the extra-articular distal humerus plate 194 may include twenty-four of the openings 150, to about 103 mm, in which the extra-articular distal humerus plate 194 may include six of the openings 150.

Similar to the medial distal humerus plate 132, the extra-articular distal humerus plate 194 may be configured to support variable positioning of the fasteners 108 and dynamic compression of the humerus 106 (or portions thereof). Accordingly, in the implementation shown in FIG. 11, starting from the second-most proximal of the first openings 150a and moving distally, every fourth of the first openings 150a includes an oblong profile. In other implementations, however, others of the openings 150 may include an oblong profile.

Also similar to the medial distal humerus plate 132, the openings 150 of the extra-articular distal humerus plate 194 may each define the fastener insertion axis 160 (e.g., the nominal fastener insertion axis) to target certain regions of the humerus 106 while avoiding other regions and surgical hardware. Accordingly, in some implementations, at least some of the openings 150 located on the shaft portion 146 may define the fastener insertion axis 160 to be substantially normal to the second surface 144 of the extra-articular distal humerus plate 194 such that the fasteners 108 have a substantially transverse trajectory into the humerus 106. The openings 150 located on the distal portion 148 may each define the fastener insertion axis 160 to target the capitellum 126 and/or other regions of the humerus 106. The openings 150 of the extra-articular distal humerus plate 194 may also each define of the fastener insertion axis 160 so as to avoid collisions between the fasteners 108. Accordingly, each of the openings 150 may define the fastener insertion axis 160 such that it does not intersect the fastener insertion axis 160 of any other of the openings 150 of the extra-articular distal humerus plate 194.

Still referring to FIG. 11, to support provisional fixation of the extra-articular distal humerus plate 194 to the humerus 106, the extra-articular distal humerus plate 194 may include the apertures 166 (e.g., seven of the apertures 166) configured to receive respective ones of the stabilizers 168. The extra-articular distal humerus plate 194 shown in FIG. 11 includes five of the apertures 166 along the shaft portion 146, in which one of the apertures 166 is each positioned at the beveled portion 172 of the proximal end 136, between the fourth and fifth-most proximal of the openings 150, between the eighth and ninth-most proximal of the openings 150, between the eleventh and twelfth-most proximal of the openings 150, and between the fourteenth and fifteenth-most proximal of the openings 150. The extra-articular distal humerus plate 194 may also include two of the apertures 166 on the distal portion 148. In other implementations, however, the apertures 166 may be positioned along the extra-articular distal humerus plate 194 according to some other configuration.

Furthermore, to mitigate edge-related post-operative irritation of adjacent soft tissue, the second surface 144 of the extra-articular distal humerus plate 194 may include the beveled portion 172 on each of the proximal end 136 and the distal end 138. The beveled portion 172 of the distal end 138 may be configured to sit on a posterolateral side of the lateral epicondyle 122, and the beveled portion 172 of the proximal end 136 may be configured to sit on a lateral side of the diaphysis 116. The beveled portion 172 of the proximal end 136 may also mitigate intra-operative irritation of soft tissue during installation of the extra-articular distal humerus plate 194 by smoothly sliding underneath soft tissue where a surgeon chooses to insert the extra-articular distal humerus plate 194 proximally from a distal location of the arm.

To reduce the amount of bending required to fit the extra-articular distal humerus plate 194 to the distal aspect 104 of the humerus 106, the extra-articular distal humerus plate 194 may include an anatomic contour configured to follow the best approximation of average distal humerus anatomy. In particular, the first surface 142 of the extra-articular distal humerus plate 194 may include an anatomic contour that substantially matches the surface 110 of the posterolateral and/or lateral side of each of the lateral epicondyle 122 of the distal epiphysis 120, the distal metaphysis 118, and/or a portion of the diaphysis 116 of an average humerus. Accordingly, the first surface 142 of the extra-articular distal humerus plate 194 along the shaft portion 146 may include an anatomic contour that substantially matches the surface 110 of the posterolateral and/or lateral side of each of the diaphysis 116, the distal metaphysis 118, and/or portions thereof. Furthermore, the first surface 142 of the extra-articular distal humerus plate 194 along the distal portion 148 may include an anatomic contour that substantially matches the surface 110 of the posterolateral side of each of the lateral epicondyle 122 of the distal epiphysis 120, the distal metaphysis 118, and/or portions thereof.

Additionally, to reduce the contact area between the first surface 142 of the extra-articular distal humerus plate 194 and the surface 110 of the humerus 106—and thereby preserve blood supply to the humerus 106—the extra-articular distal humerus plate 194 may include the undercuts (not shown) spaced apart from one another along the first surface 142 of the extra-articular distal humerus plate 194.

### Proximal Ulna Stabilization System

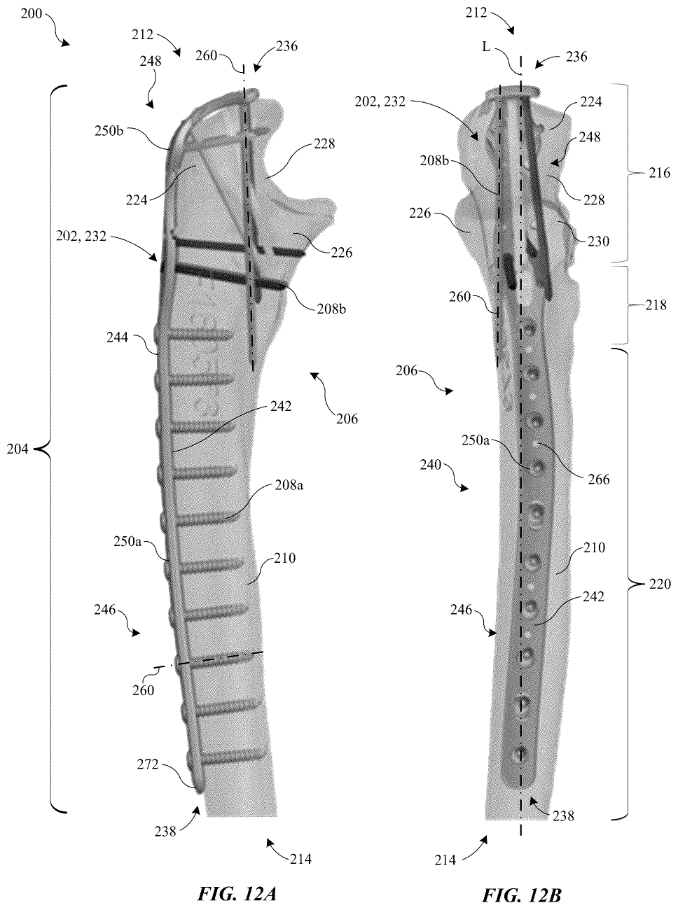

FIG. 12 shows a proximal ulna stabilization system 200 according to a first example that utilizes a first example of a proximal ulna plate 202 to stabilize a proximal aspect 204 of an ulna 206. Implementations of the proximal ulna plate 202 described herein are configured for internal fixation of the ulna 206, in which the proximal ulna plate 202 is secured directly to the ulna 206 via fasteners 208a and 208b collectively referred to as fasteners 208 (e.g., bone screws) such that the proximal ulna plate 202 sits on (e.g., contacts) a surface 210 of the ulna 206 while being positioned beneath adjacent soft tissue. By securing the proximal ulna plate 202 directly to the surface 210 of the ulna 206, load may be transferred from the ulna 206 to the proximal ulna plate 202 to support the ulna 206 in a fixed position while maintaining fracture reduction during healing. Internal fixation may be utilized to treat a variety of injuries and conditions where controlled (e.g., supported) healing of bone may be desired.

With respect to internal fixation of the proximal aspect 204 of the ulna 206, some such injuries may include intra-articular or extra-articular proximal ulna fractures, periprosthetic fractures, nonunion or malunion repairs, proximal ulna fractures that include osteopenic bone, olecranon osteotomies, commuted proximal ulna fractures, multi-fragmentary proximal ulna fractures, and distally extending proximal ulna fractures. Although a handful of applicable injuries are described herein, other injuries and conditions benefitted by controlled healing of the proximal aspect 204 of the ulna 206 are also contemplated by this disclosure. As will become apparent, the implementations of the proximal ulna plate 202 may enable the use of a single bone plate to treat such injuries and conditions where multiple bone plates may otherwise be required.

The ulna 206 is a long bone in the arm or forelimb that runs from the elbow to the wrist. Long bones may be generally described with respect to a proximal end 212 and a distal end 214 thereof, as well as with respect to regions of the long bone segmented along a longitudinal axis L. Beginning from the proximal end 212, the regions include a proximal epiphysis 216, a proximal metaphysis 218, a diaphysis 220, a distal metaphysis (not shown), and a distal epiphysis (not shown). Accordingly, the proximal aspect 204 of the ulna 206—located toward the elbow—may include the proximal epiphysis 216, the proximal metaphysis 218, and a portion of the diaphysis 220. Furthermore, a distal aspect of the ulna 206—located toward the wrist—may include the distal epiphysis, the distal metaphysis, and another portion of the diaphysis 220. Long bones also generally include an intermedullary canal 222 that extends longitudinally through a center of the long bone. Additionally, the proximal epiphysis 216 of the ulna 206 may be described with respect to regions thereof, including an olecranon 224, a coronoid 226, a trochlear notch 228, and a radial notch 230. Although generally described with reference to the ulna 206, it will be appreciated that the proximal ulna stabilization system 200 described herein may be used or adapted to be used for internal fixation of other long bones as well, such as the femur, radius, tibia, etc. Furthermore, although the ulna 206 is generally depicted as a human ulna bone, it will be appreciated that the proximal ulna stabilization system 200 described herein may also be used or adapted to be used for internal fixation of long bones of other mammals as well, such as dogs, cats, horses, etc.

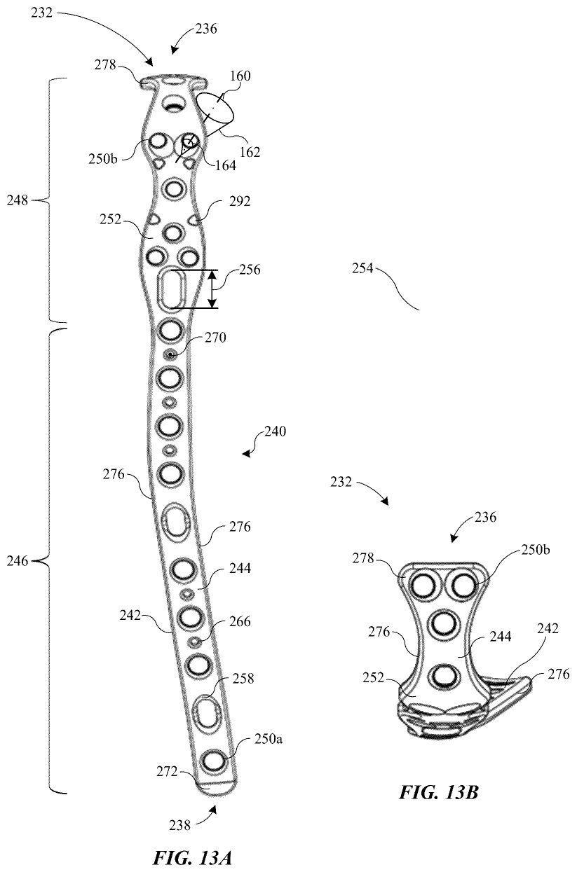

Implementations of the proximal ulna plate 202 described herein are configured to sit on a posterior side of the olecranon 224 of the ulna 206. However, some implementations of the proximal ulna plate 202 may extend longitudinally along the ulna 206 to different extents and may be configured to treat different injuries and/or conditions. The first example of the proximal ulna plate 202 shown in FIGS. 12A-13B, for example, is configured to extend along the ulna 206 to a highly proximal location of the olecranon 224. Accordingly, the first example of the proximal ulna plate 202 may be referred to as a very proximal ulna plate 232. Although many of the features of the proximal ulna plate 202 are described with reference to the very proximal ulna plate 232, it will be appreciated that other implementations of the proximal ulna plate 202 described herein may include features similar to those of the very proximal ulna plate 232, except where expressly indicated.

Referring now to FIGS. 12A-13B, the very proximal ulna plate 232 extends longitudinally along the posterior side of the proximal aspect 204 to define a proximal end 236, a distal end 238, and a length 240 extending therebetween. The proximal end 236 may be configured to be positioned along the posterior side of the diaphysis 220 of the ulna 206. The distal end 238 may be configured to be positioned along the posterior side of the proximal epiphysis 216 of the ulna 206 and may be configured to be positioned along the olecranon 224 of the proximal epiphysis 216. The very proximal ulna plate 232 includes a first surface 242 configured to contact the surface 210 of the ulna 206 and an opposite, second surface 244.

The very proximal ulna plate 232 may include a shaft portion 246 that is configured to extend along the diaphysis 220, the proximal metaphysis 218, and/or portions thereof. The very proximal ulna plate 232 may also include a proximal portion 248 that is configured to extend along the proximal epiphysis 216, the proximal metaphysis 218, and/or portions thereof. In other words, the shaft portion 246 may correspond to at least one of the diaphysis 220 or the proximal metaphysis 218 of the ulna 206, and the proximal portion 248 may correspond to at least one of the proximal epiphysis 216 or the proximal metaphysis 218 of the ulna 206. As shown in FIGS. 12A-13B, the proximal portion 248 may include a series of bulbus regions 252 (e.g., two of the bulbus regions 252) spaced longitudinally along the proximal portion 248 to terminate at a triangular region 278 located at the proximal end 236 of the very proximal ulna plate 232. Accordingly, sides 276 of the very proximal ulna plate 232 may curve arcuately outward at each of the bulbus regions 252 and curve arcuately outward at the triangular region 278 before terminating at the proximal end 236 of the very proximal ulna plate 232.

Still referring to FIGS. 12A-13B, the very proximal ulna plate 232 may include first openings 250a and second openings 250b, collectively referred to as openings 250 (e.g., twenty-two of the openings 250) that are configured to receive the fasteners 208. The fasteners 208 may include locking fasteners, non-locking fasteners, or any other suitable fasteners known in the art. In some implementations, the fasteners 208 may include bone screws, such as fixed or variable angle bone screws. The fasteners 208 may also include other fasteners or features configured to secure to or engage with bone, such as nails, pikes, staples, pegs, barbs, hooks, or the like. The fasteners 208 may include a shaft portion configured to engage bone and a head portion configured to engage the very proximal ulna plate 232. Although the very proximal ulna plate 232 shown in FIGS. 12A-13B comprises twenty-two of the openings 250, longer and shorter implementations of the very proximal ulna plate 232 having different quantities and configurations of the openings 250 are also contemplated by this disclosure.

The openings 250 extend through the very proximal ulna plate 232 from the first surface 242 to the second surface 244. The openings 250 may be spaced apart from one another along the length 240 of the very proximal ulna plate 232. As shown in FIG. 12A-13B, at least some of the openings 250 located on the shaft portion 246 may be spaced apart from one another longitudinally along a center line 254 that longitudinally bisects the very proximal ulna plate 232 along the length 240. The openings 250 located on the proximal portion 248, however, may have some other configuration. For example, some of the openings 250 may be positioned along the center line 254 and some of the openings 250 may be spaced laterally (e.g., horizontally) adjacent to one another on either side of the center line 254. In the implementation shown in FIGS. 12A-13B, the first and second-most proximal of the openings 250 (located on the triangular region 278), the fifth and sixth-most proximal of the openings 250 (located on one of the bulbus regions 252), and ninth and tenth-most proximal of the openings 250 (located on the other of the bulbus regions 252) are positioned horizontally adjacent to one another. All other of the openings 250 located on the proximal portion 248 may be positioned along the center line 254. The very proximal ulna plate 232, however, may include any suitable number of the openings 250 in any suitable configuration. Surgeons may desire flexibility for placement of the fasteners 208 based on preference, anatomy, and fracture location. Furthermore, surgeons may have differing opinions as to the number, location, and types of the fasteners 208 used to treat a given injury. Accordingly, the openings 250 described herein may provide surgeons a variety of options for securing the very proximal ulna plate 232 to the ulna 206.

For example, it may be desired that the fasteners 208 be configured to lock to the very proximal ulna plate 232 after installation. Accordingly, one or more of the openings 250 may include locking features that interface with corresponding features of the fasteners 208 to form a locking engagement therebetween. For example, where the fasteners 208 include bone screws, the one or more of the openings 250 may include threads, texture (e.g., knurling), or the like which form a locking engagement with corresponding features of the fasteners 208 (e.g., corresponding threads, texture, or the like on the head portion of the fasteners 208) after being inserted through the openings 250 and tightened.

Variable positioning of the fasteners 208 relative to the very proximal ulna plate 232 may also be desired. Accordingly, one or more of the openings 250 may include an oblong profile that defines a length 256 along which the fasteners 208 may be positioned. By enabling variable positioning of the fasteners 208 along the length 256 of the oblong profile, the very proximal ulna plate 232 may be movable relative to the ulna 206 (or fragments thereof) after installation (e.g., partial installation) of the fasteners 208. In some implementations, the length 256 of the oblong profile may be about 6 mm. Furthermore, the oblong profile of the one or more openings 250 may include ramped surfaces 258 on each end thereof to support dynamic compression of the ulna 206, which may be desired to treat certain fracture types (e.g., transverse fractures, etc.). To support dynamic compression, some of the fasteners 208 may include a rounded surface on the head portion that is configured to slide against the ramped surfaces 258 of the openings 250 to dynamically compress regions of the ulna 206 along the length 240 of the very proximal ulna plate 232 upon tightening of the fasteners 208. It will be recognized by those skilled in the art that dynamic tensioning can also be achieved using the described configuration.

Additionally, it may be desired that the fasteners 208 target certain regions of the ulna 206, such as fragmented regions of the ulna 206. In the case of highly comminuted fractures, targeting fragmented regions of the ulna 206 may enable a surgeon to achieve “absolute stability” of the proximal aspect 204 in which healing may occur with minimal callus formation between bone fragments. Furthermore, it may be desired that the fasteners 208 target regions of the ulna 206 that are comprised of high quality (e.g., dense and/or healthy) bone to achieve a high degree of bone purchase. It may also be desired that the fasteners 208 avoid certain regions of the ulna 206 or other surgical hardware. For example, it may be desired that the fasteners 208 avoid regions of the ulna 206 comprised of low quality (e.g., soft and/or compromised) bone. As another example, it may be desired that the fasteners 208 avoid other of the fasteners 208 that are associated with the very proximal ulna plate 232. As yet another example, it may be desired that the fasteners 208 avoid other surgical hardware that may be installed in the ulna 206 such as orthopedic implants and the like.

Accordingly, the openings 250 may be configured to aid surgeons in targeting and/or avoiding regions of the ulna 206 by defining a fastener insertion axis 260 (e.g., a trajectory) for each of the fasteners 208 to be inserted therethrough and into the ulna 206. For example, one or more of the openings 250 may be substantially cylindrical such that the fastener insertion axis 260 aligns with an axis of the cylindrical opening. As described above, however, surgeons may desire flexibility for placement of the fasteners 208 based on several factors. Therefore, to provide such flexibility, one or more of the openings 250 may be substantially conical (e.g., frustoconical) such as to provide surgeons a conical range of fastener insertion axes 262 for the fasteners 208 to be inserted therethrough. In such an implementation, the fastener insertion axis 260 may be referred to as a nominal fastener insertion axis and the conical range of fastener insertion axes 262 may surround the nominal fastener insertion axis. In some implementations, the conical range of fastener insertion axes 262 may define a cone having an aperture angle 264. In some implementations, the aperture angle 264 of the conical range of fastener insertion axes 262 may be about 20-degrees, 40-degrees, 60-degrees, or some other angle.

In some implementations, at least some of the openings 250 located on the shaft portion 246 may each define the fastener insertion axis 260 (e.g., the nominal fastener insertion axis) to be substantially normal to the second surface 244 of the very proximal ulna plate 232 such that the fasteners 208 have a substantially transverse trajectory into the ulna 206. Some of the openings 250 located on the shaft portion 246, however, may define the fastener insertion axis 260 to aim proximally, distally, or at some other angle relative to the ulna 206. The openings 250 located on the proximal portion 248 may each define the fastener insertion axis 260 (e.g., the nominal fastener insertion axis) to target the coronoid 226, the olecranon 224, the intermedullary canal 222, and/or other regions of the proximal aspect 204 of the ulna 206.

For example, as best shown in FIGS. 12A-12B, the first and second-most proximal of the openings 250 (located laterally adjacent to one another on the triangular region 278) may each define the fastener insertion axis 260 to aim distally along the longitudinal axis L of the ulna 206 to target the diaphysis 220 and/or the proximal metaphysis 218. The third-most proximal of the openings 250 (also located on the triangular region 278) and the fourth-most proximal of the openings 250 (located on one of the bulbus regions 252) may each define the fastener insertion axis 260 to aim transversely and distally toward the coronoid 226 and/or the anterior side of the proximal metaphysis 218. The fifth and sixth-most proximal of the openings 250 (located laterally adjacent to one another on the one of the bulbus regions 252) may each define the fastener insertion axis 260 to aim transversely and proximally toward a proximal region of the olecranon 224. The eighth-most proximal of the openings 250 (located on the other of the bulbus regions 252) and the ninth and tenth-most proximal of the openings 250 (located laterally adjacent to one another also on the other of the bulbus regions 252) may each define the fastener insertion axis 260 to aim transversely toward the coronoid 226 and/or the anterior side of the proximal metaphysis 218.

Furthermore, to avoid collisions during installation of the fasteners 208, each of the openings 250 may define the fastener insertion axis 260 (e.g., the nominal fastener insertion axis) such as to not intersect the fastener insertion axis 260 of any other of the openings 250 of the very proximal ulna plate 232. For example, as best shown in FIG. 12B, one of the two-most proximal of the openings 250 (located on the triangular region 278) may define the fastener insertion axis 260 to aim distally along the longitudinal axis L toward a medial side of the diaphysis 220, and the other of the two-most proximal of the openings 250 (also located on the triangular region 278) may each define the fastener insertion axis 260 to aim distally along the longitudinal axis L toward a lateral side of the proximal metaphysis 218. The fasteners 208 that are inserted along such a trajectory are often referred to as columnar fasteners and may be used to reduce and provide compression to transverse fractures, highly comminuted and irregular fractures, and the like. Surgeons often express difficulty when maneuvering other fasteners past such columnar fasteners. Thus, the fastener insertion axis 260 of each of the openings 250 may be carefully defined such as to avoid such collisions.

Still referring to FIGS. 12A-13B, the fasteners 208 may comprise the first fasteners 208a and the second fasteners 208b, in which the first fasteners 208a have a larger diameter than the second fasteners 208b. Accordingly, the openings 250 may comprise the first openings 250a and the second openings 250b, in which the first openings 250a have a larger diameter than the second openings 250b. In some implementations, the first fasteners 208a include 3.5 mm bone screws, and the second fasteners 208b include 2.5 mm bone screws. To provide robust fixation of the very proximal ulna plate 232 to the ulna 206, the first fasteners 208a may be desired along the shaft portion 246 to secure the very proximal ulna plate 232 to the diaphysis 220 and/or the proximal metaphysis 218 of the ulna 206.

Accordingly, at least one of the first openings 250a (e.g., ten of the first openings 250a) configured to receive the first fasteners 208a may be positioned along the shaft portion 246. Furthermore, at least one of the first openings 250a (e.g., one of the first openings 250a) may be positioned along the proximal portion 248. Some of the first openings 250a (e.g., two of the first openings 250a) may include an oblong profile as described above. In the implementation shown in FIGS. 12A-13B, the second-most distal of the first openings 250a, the sixth-most distal of the first openings 250a, and the eleventh-most distal of the first openings 250a may include an oblong profile. In other implementations, however, others of the openings 250 may include an oblong profile. On the other hand, to target specific regions of the proximal aspect 204 of the ulna 206, the second fasteners 208b may be desired along the proximal portion 248 to target the coronoid 226, the olecranon 224, the intermedullary canal 222, and/or other regions of the proximal aspect 204 of the ulna 206.

To aid surgeons in the installation of the very proximal ulna plate 232 to the ulna 206, the very proximal ulna plate 232 may include apertures 266 (e.g., five of the apertures 266) that are configured to receive respective stabilizers 268. The apertures 266 may generally have a smaller diameter than the openings 250. The stabilizers 268 may be inserted through respective ones of the apertures 266 to provisionally fix the very proximal ulna plate 232 to the ulna 206 prior to installation of the fasteners 208. In some implementations, the stabilizers 268 may include a K-wire. In other implementations, the stabilizers 268 may include pins or the like. The apertures 266 extend through the very proximal ulna plate 232 from the first surface 242 to the second surface 244. At least some of the apertures 266 may be adjacent to one or more of the openings 250. Thus, in the implementation shown in FIGS. 12A-13B where some of the openings 250 are positioned along the center line 254, some of the apertures 266 may also be positioned along the center line 254. In some implementations, five of the apertures 266 may be positioned along the shaft portion 246, in which one of the apertures 266 is each positioned between the third and fourth-most distal of the openings 250, between the fourth and fifth-most distal of the openings 250, between the seventh and eighth-most distal of the openings 250, between the eighth and ninth-most distal of the openings 250, and between the ninth and tenth-most distal of the openings 250. In other implementations, however, the apertures 166 may be positioned along the medial distal humerus plate 132 according to some other configuration.

The apertures 266 may each define a stabilizer insertion axis 270 (e.g., a trajectory) for the stabilizers 268 to be inserted therethrough and into the ulna 206. In some implementations, at least some of the apertures 266 may define the stabilizer insertion axis 270 to be substantially parallel with the fastener insertion axis 260 of the one or more openings 250 to which a respective one of the apertures 266 is adjacent. By defining the stabilizer insertion axis 270 to be substantially parallel with the fastener insertion axis 260 of adjacent ones of the openings 250, surgeons may be able to acquire a sense of the actual trajectory of the fasteners 208 into the ulna 206 prior to installation of the fasteners 208 by provisional installation of the stabilizers 268.

The very proximal ulna plate 232 may also include suture holes 292 (e.g., four of the suture holes 292) to enable provisional fixation of the very proximal ulna plate 232 to adjacent soft tissue. The suture holes 292 may also facilitate repair of adjacent soft tissue in conjunction with installation of the very proximal ulna plate 232. The suture holes 292 extend from the first surface 242 to the second surface 244 of the very proximal ulna plate 232. In some implementations, the suture holes 292 may be substantially triangular, however, other geometries may be used. As shown in FIG. 13A, the very proximal ulna plate 232 may include two of the suture holes 292 positioned laterally (e.g., horizontally) adjacent to one another between the fifth and sixth-most proximal and the seventh-most proximal of the openings 250 (on one of the bulbus regions 252). The very proximal ulna plate 232 may also include two of the suture holes 292 positioned laterally (e.g., horizontally) adjacent to one another between the seventh-most proximal and the eighth-most proximal of the openings 250 (on the other of the bulbus regions 252). In other implementations, however, the suture holes 292 may be positioned at other locations along the very proximal ulna plate 232.

Edges and other protrusions and irregularities of the very proximal ulna plate 232 may irritate adjacent soft tissue post-operatively, especially around regions of the ulna 206 that have minimal adjacent soft tissue such as the olecranon 224. Therefore, to mitigate soft tissue irritation post-operatively, the head portion of the fasteners 208 may be configured to be received by the openings 250 such that the head portion sits flush or recessed with respect to the second surface 244 of the very proximal ulna plate 232. Additionally, the second surface 244 of the very proximal ulna plate 232 may be beveled at the proximal end 236 to form a beveled portion 272. In some implementations, a periphery (e.g., or portions thereof) of the beveled portion 272 may be radiused (e.g., may include a radius of about 0.5 mm). By softening the edge of the very proximal ulna plate 232 at the proximal end 236, soft tissue irritation around the olecranon 224 may be mitigated or prevented.

Furthermore, surgeons may choose to install the very proximal ulna plate 232 by inserting the plate through soft tissue of an arm from a proximal location along the arm such that the very proximal ulna plate 232 slides under the soft tissue as it travels distally toward the distal end 114 of the ulna 206. When inserting the very proximal ulna plate 232 beneath the triceps insertion near the elbow, the proximal portion 248 may irritate or damage (e.g., split) the triceps tendon and/or surrounding tissue. To mitigate or eliminate such irritation or damage, the proximal portion 248 may be thinner than the shaft portion 246. In some implementations, the thickness of the proximal portion 248 may gradually thin (e.g., taper) toward the proximal end 236 of the very proximal ulna plate 232. The shape of the proximal portion 248 (e.g., the sides 276 curving arcuately along the proximal portion 248) may also mitigate irritation or damage to the triceps tendon and/or surrounding tissue.

Due to the complex geometry of the proximal aspect 204 of the ulna 206, especially with respect to the proximal epiphysis 216 and the proximal metaphysis 218, surgeons are often required to bend bone plates to some extent, pre-operatively and/or intra-operatively, to contour the bone plate to the proximal aspect 204 of the ulna 206. Thus, to reduce the amount of bending required (or eliminate the need for bending altogether), the very proximal ulna plate 232 may include an anatomic contour configured to follow the best approximation of an average proximal ulna anatomy. In particular, the first surface 242 of the very proximal ulna plate 232 may include an anatomic contour that substantially matches the surface 210 of the posterior side of the olecranon 224 of the proximal epiphysis 216, the proximal metaphysis 218, and/or a portion of the diaphysis 220 of an average ulna. Accordingly, the first surface 242 of the very proximal ulna plate 232 along the shaft portion 246 may include an anatomic contour that substantially matches the surface 210 of the posterior side of each of the diaphysis 220, the proximal metaphysis 218, and/or portions thereof. Furthermore, the first surface 242 of the very proximal ulna plate 232 along the proximal portion 248 may include an anatomic contour that substantially matches the surface 210 of the posterior side of each of the olecranon 224 of the proximal epiphysis 216, the proximal metaphysis 218, and/or portions thereof.

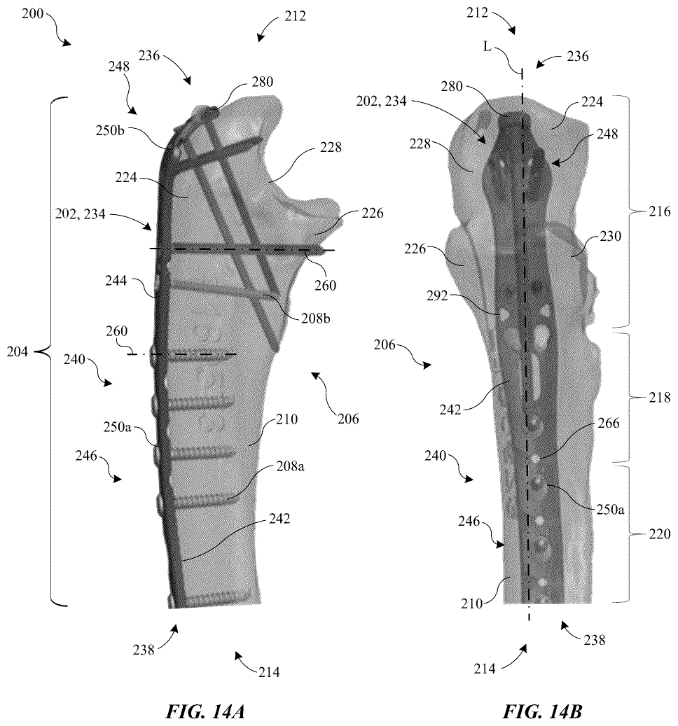

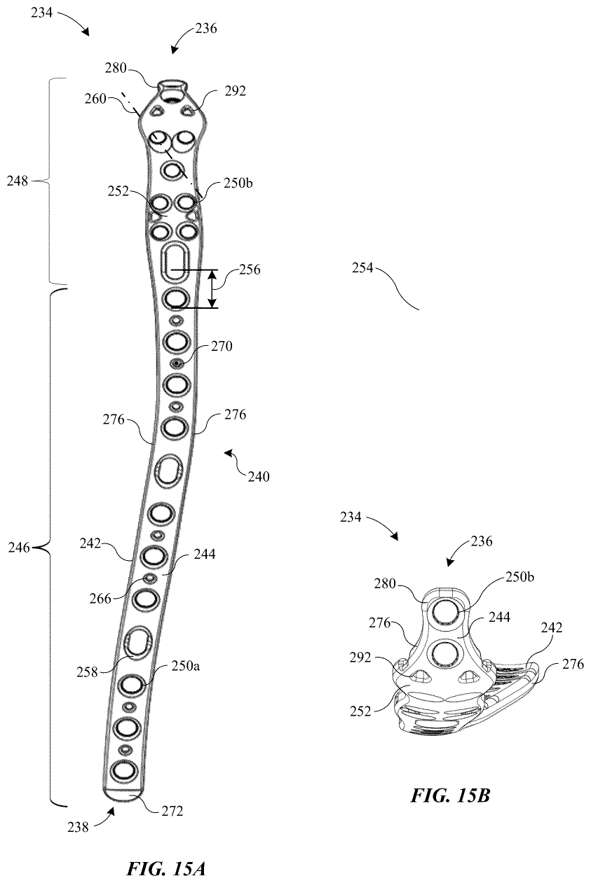

FIGS. 14A-15B show a second example of the proximal ulna plate 202. The second example of the proximal ulna plate 202 is similar to the very proximal ulna plate 232 except that the second example of the proximal ulna plate 202 does not extend as proximally along the olecranon 224 as the very proximal ulna plate 232. Accordingly, the second example of the proximal ulna plate 202 may be referred to a less proximal ulna plate 234. The proximal portion 248 of the less proximal ulna plate 234 may also include a series of the bulbus regions 252 (e.g., two of the bulbus regions 252) spaced longitudinally along the proximal portion 248 to terminate at a narrow region 280 located at the proximal end 236 of the less proximal ulna plate 234. Accordingly, sides 276 of the very proximal ulna plate 232 may curve arcuately outward at each of the bulbus regions 252 and curve arcuately inward at the narrow region 280 before terminating at the proximal end 236 of the less proximal ulna plate 234.

The less proximal ulna plate 234 shown in FIGS. 14A-15B comprises twenty-two of the openings, including thirteen of the first openings 250a and nine of the second openings 250b. Twelve of the first openings 250a may be spaced apart from one another longitudinally along the shaft portion 246 of the less proximal ulna plate 234 (e.g., along the center line 254), and one of the first openings 250a may be located on the proximal portion 248. The nine of the second openings 250b may be spaced apart from one another along the proximal portion 248. Some of the openings located on the proximal portion 248 may be positioned along the center line 254 and some of the openings 250 may be spaced laterally (e.g., horizontally) adjacent to one another on either side of the center line 254. In the implementation shown in FIG. 14A-15B, the third and fourth-most proximal of the openings 250 (located on one of the bulbus regions 252), the fifth and sixth-most proximal of the openings 250 (located on the other of the bulbus regions 252), and the seventh and eighth-most proximal of the openings (also located on the other of the bulbus regions 252) are positioned laterally (e.g., horizontally) adjacent to one another. All other of the openings 250 located on the proximal portion 248 may be positioned along the center line 254. Although the less proximal ulna plate 234 shown in FIGS. 14A-15B comprises twenty-two of the openings 250, longer and shorter length implementations of the less proximal ulna plate 234 having different quantities and configurations of the openings 250 are also contemplated by this disclosure.

Similar to the very proximal ulna plate 232, the less proximal ulna plate 234 may be configured to support variable positioning of the fasteners 208 and dynamic compression of the ulna 206 (or portions thereof). Accordingly, in the implementation shown in FIGS. 14A-15B, beginning at the distal end 238 of the less proximal ulna plate 234 and moving proximally, every fourth of the first openings 250a may include an oblong profile. In other implementations, however, others of the openings 250 may include an oblong profile.

The openings 250 of the less proximal ulna plate 234 may each define the fastener insertion axis 260 (e.g., the nominal fastener insertion axis) to target certain regions of the ulna 206 while avoiding other regions and surgical hardware. Accordingly, in some implementations, at least some of the openings 520 located on the shaft portion 426 may define the fastener insertion axis 260 to be substantially normal to the second surface 244 of the less proximal ulna plate 234 such that the fasteners 208 have a substantially transverse trajectory into the ulna 206. The openings 250 located on the proximal portion 248 may each define the fastener insertion axis 260 to target the coronoid 226, the olecranon 224, the intermedullary canal 222 and/or other regions of the proximal aspect 204 of the ulna 206.

For example, as best shown in FIGS. 14A-14B, the first-most proximal of the openings 250 (located on the narrow region 280) and the second-most proximal of the openings 250 (also located on the narrow region 280) may each define the fastener insertion axis 260 to aim transversely and distally toward the coronoid 226. The third and fourth-most proximal of the openings 250 (located laterally adject to one another on one of the bulbus regions 252) may each define the fastener insertion axis 260 to aim transversely and proximally toward a proximal region of the olecranon 224. The sixth and seventh-most proximal of the openings 250 (located laterally adjacent to one another on the other of the bulbus regions 252) and the eighth and ninth-most proximal of the openings 250 (also located laterally adjacent to one another on the other of the bulbus regions 252) may each define the fastener insertion axis 260 to aim transversely toward the coronoid 226.

Still referring to FIGS. 14A-15B, to support provisional fixation of the less proximal ulna plate 234 to the ulna 206, the less proximal ulna plate 234 may include the apertures 266 (e.g., seven of the apertures 266) configured to receive respective ones of the stabilizers 268. The less proximal ulna plate 234 shown in FIGS. 14A-15B includes seven of the apertures 266 along the shaft portion 246, in which one of the apertures 266 is positioned between the first and second-most distal of the openings 250, between the second and third-most distal of the openings 250, between the fifth and sixth-most distal of the openings 250, between the sixth and seventh-most distal of the openings 250, between the ninth and tenth-most distal of the openings 250, between the tenth and eleventh-most distal of the openings 250, and between the eleventh and twelfth-most distal of the openings 250. In other implementations, however, the apertures 266 may be positioned along the less proximal ulna plate 234 according to some other configuration.

The less proximal ulna plate 234 may also include the suture holes 292 (e.g., four of the suture holes 292) to enable provisional fixation of the less proximal ulna plate 234 to adjacent soft tissue. As shown in FIG. 13A, the less proximal ulna plate 234 may include two of the suture holes 292 positioned laterally (e.g., horizontally) adjacent to one another between the second most proximal and the third and fourth-most proximal of the openings 250 (on one of the bulbus regions 252). The less proximal ulna plate 234 may also include two of the suture holes 292 positioned laterally (e.g., horizontally) adjacent to one another between the sixth and seventh-most proximal and the eighth and ninth-most proximal of the openings 250 (on the other of the bulbus regions 252). In other implementations, however, the suture holes 292 may be positioned at other locations along the less proximal ulna plate 234.

Furthermore, to mitigate edge-related intra-operative irritation and damage to the triceps insertion and surrounding tissue where a surgeon chooses to insert the less proximal ulna plate 232 beneath the triceps insertion near the elbow, the proximal portion 248 may be thinner than the shaft portion 246. In some implementations, the thickness of the proximal portion 248 may gradually thin (e.g., taper) toward the proximal end 236 of the less proximal ulna plate 234. The shape of the proximal portion 248 (e.g., the sides 276 curving arcuately along the proximal portion 248) may also mitigate irritation or damage to the triceps tendon and/or surrounding tissue.

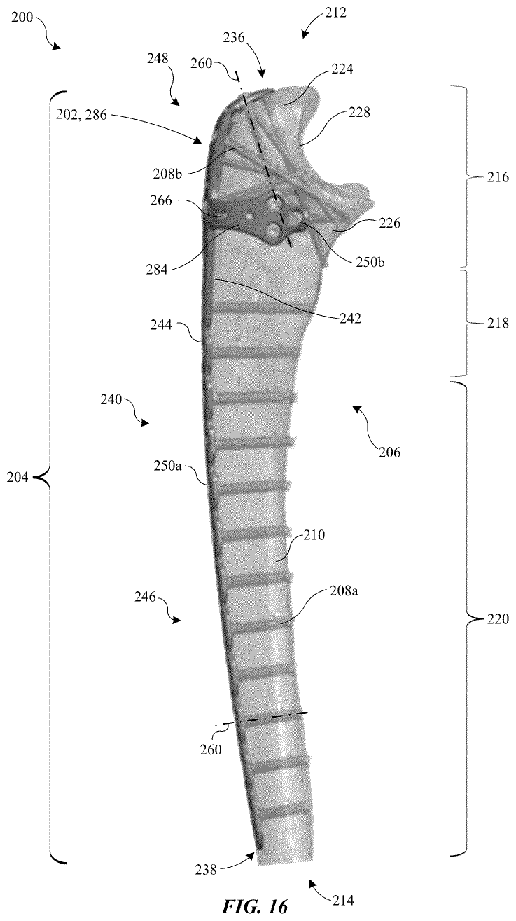

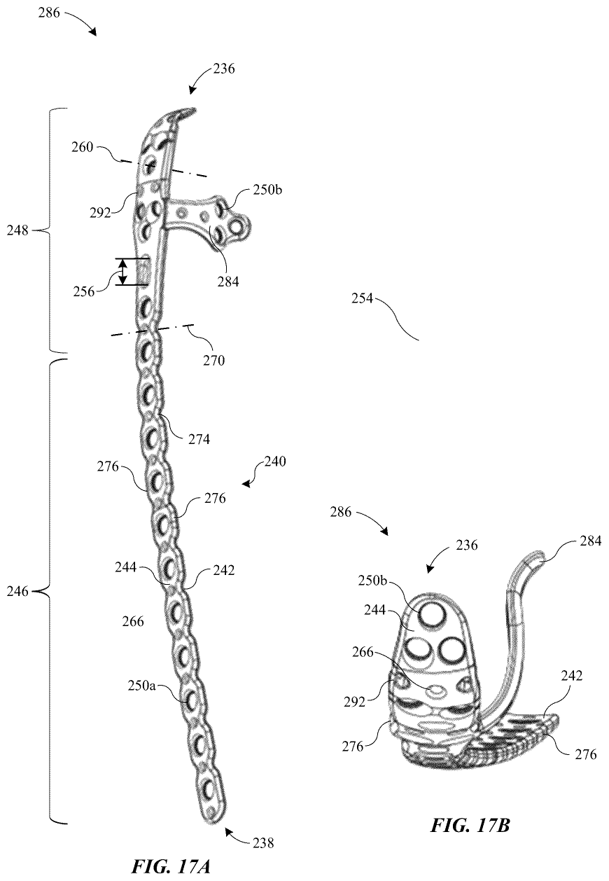

FIGS. 16-17B show a third example of the proximal ulna plate 202. The third example of the proximal ulna plate 202 is similar to the very proximal ulna plate 232 except that the third example of the proximal ulna plate 202 does not extend as proximally along the olecranon 224 as the very proximal ulna plate 232 and includes a medial extension plate 284 that extends medially from the proximal portion 248. Accordingly, the third example of the proximal ulna plate 202 may be referred to a medially extending proximal ulna plate 286. The proximal portion 248 of the less proximal ulna plate 234 may exclude the bulbus regions 252 and the triangular region 278 of the very proximal ulna plate 232. Instead, the width of the medially extending proximal ulna plate 286 between the sides 276 may gradually narrow (e.g., taper) toward the proximal end 236.

The medially extending proximal ulna plate 286 shown in FIGS. 16-17B comprises twenty-five of the openings, including twelve of the first openings 250a spaced apart from one another longitudinally along the shaft portion 246 (e.g., along the center line 254), and thirteen of the second openings 250b spaced apart from one another along the proximal portion 248 and the medial extension plate 284. Some of the openings located on the proximal portion 248 may be positioned along the center line 254 and some of the openings 250 may be spaced laterally (e.g., horizontally) adjacent to one another on either side of the center line 254. In the implementation shown in FIG. 16-17B, the second and third-most proximal of the openings 250, the fourth and fifth-most proximal of the openings 250, and the seventh and eighth-most proximal of the openings of the proximal portion 248 are positioned laterally (e.g., horizontally) adjacent to one another. All other of the openings 250 located on the proximal portion 248 may be positioned along the center line 254.

Furthermore, as described above the proximal portion 248 of the medially extending proximal ulna plate 286 may include the medial extension plate 284 that extends generally in medial direction toward the coronoid 226 from the proximal portion 248 to sit on a medial (e.g., posteromedial) side of the olecranon 224 of the ulna 206. Accordingly, the first surface 242 of the medially extending proximal ulna plate 286 may include an anatomical contour that substantially matches the surface 210 of a medial (posteromedial) side of the olecranon 224. The medial extension plate 284 may include three of the second openings 250b. In some implementations, two of the second openings 250b of the medial extension plate 284 may be spaced longitudinally (e.g., vertically) adjacent to one another. Although the less proximal ulna plate shown in FIGS. 14A-15B comprises twenty-two of the openings 250, longer and shorter length implementations of the less proximal ulna plate 234 having different quantities and configurations of the openings 250 are also contemplated by this disclosure.

Similar to the very proximal ulna plate 232, the medially extending proximal ulna plate 286 may be configured to support variable positioning of the fasteners 208 and dynamic compression of the ulna 206 (or portions thereof). Accordingly, in the implementation shown in FIGS. 16-17B, the thirteenth-most distal of the openings 250 may include an oblong profile. In other implementations, however, others of the openings 250 may include an oblong profile.

The openings 250 of the medially extending proximal ulna plate 286 may each define the fastener insertion axis 260 (e.g., the nominal fastener insertion axis) to target certain regions of the ulna 206 while avoiding other regions and surgical hardware. Accordingly, in some implementations, at least some of the openings 250 located on the shaft portion 246 may define the fastener insertion axis 260 to be substantially normal to the second surface 244 of the medially extending proximal ulna plate 286 such that the fasteners 208 have a substantially transverse trajectory into the ulna 206. The openings 250 located on the proximal portion 248 and the medial extension plate 284 may each define the fastener insertion axis 260 to target the coronoid 226, the olecranon 224, the intermedullary canal 222, and/or other regions of the proximal aspect 204 of the ulna 206.

For example, as best shown in FIG. 16, the first through sixth-most proximal of the openings 250 may each define the fastener insertion axis 260 to aim generally transversely and distally toward the coronoid 226 and/or the anterior side of the proximal metaphysis 218. The seventh and eighth-most proximal of the openings 250 (located laterally adject to one another) may each define the fastener insertion axis 260 to aim transversely toward the trochlear notch 228. The openings 250 located on the medial extension plate 284 may each define the fastener insertion axis 260 to aim transversely into the coronoid 226 from the medial (e.g., posteromedial) side of the olecranon 224.

Still referring to FIGS. 16-17B, to support provisional fixation of the medially extending proximal ulna plate 286 to the ulna 206, the medially extending proximal ulna plate 286 may include the apertures 266 (e.g., seventeen of the apertures 266) configured to receive respective ones of the stabilizers 268. The medially extending proximal ulna plate 286 shown in FIGS. 16-17B includes twelve of the apertures 266 along the shaft portion 246, in which one of the apertures 266 is positioned at the distal end 238 and one of the apertures 266 is positioned between each adjacent pair of the first openings 250a. The medially extending proximal ulna plate 286 may also include three of the apertures 266 on the proximal portion 248 and two of the apertures 266 on the medial extension plate 284. In other implementations, however, the apertures 266 may be positioned along the less proximal ulna plate 234 according to some other configuration.

The less proximal ulna plate 234 may also include the suture holes 292 (e.g., two of the suture holes 292) to enable provisional fixation of the less proximal ulna plate 234 to adjacent soft tissue. As shown in FIG. 13A, the less proximal ulna plate 234 may include two of the suture holes 292 positioned laterally (e.g., horizontally) adjacent to one another between the second and third-most proximal and the fourth and fifth-most proximal of the openings 250. In other implementations, however, the suture holes 292 may be positioned at other locations along the less proximal ulna plate 234.

Furthermore, to reduce the time and effort required to bend the medially extending proximal ulna plate 286, the medially extending proximal ulna plate 286 may include recesses 274 (e.g., scallop cuts) spaced apart from one another along the sides 276 of the medially extending proximal ulna plate 286. In some implementations, the recesses 274 may be positioned between select ones of the openings 250. For example, the medially extending proximal ulna plate 286 shown in FIGS. 16-17B includes one of the recesses 274 on each of the sides 276 between each of the first openings 250a along the shaft portion 246. In other implementations, however, the recesses 274 may be positioned along the medially extending proximal ulna plate 286 according to some other configuration.

The recesses 274 may be in the form of partially cylindrical valleys cut along the sides 276 of the medially extending proximal ulna plate 286. However, other geometries may be used. By removing material from the sides 276 of the medially extending proximal ulna plate 286 at select locations, the time and effort required to bend the medially extending proximal ulna plate 286 may be reduced without substantially compromising the structural integrity thereof. In addition to reducing the effort required to bend the medially extending proximal ulna plate 286, the recesses 274 may also discourage warping of the openings 250, the apertures 266, and/or other features of the medially extending proximal ulna plate 286 by directing bending stresses to the recesses 274. The recesses 274 may also aid in preserving blood supply to the ulna 206 (e.g., a periosteum of the ulna 206), and thereby prevent osteonecrosis, by reducing the contact area between the first surface 242 of the medially extending proximal ulna plate 286 and the surface 210 of the ulna 206.

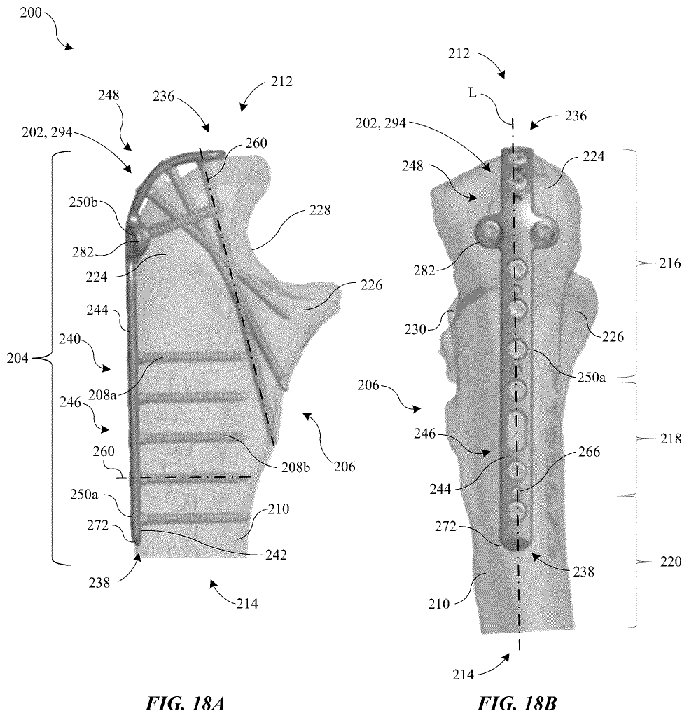

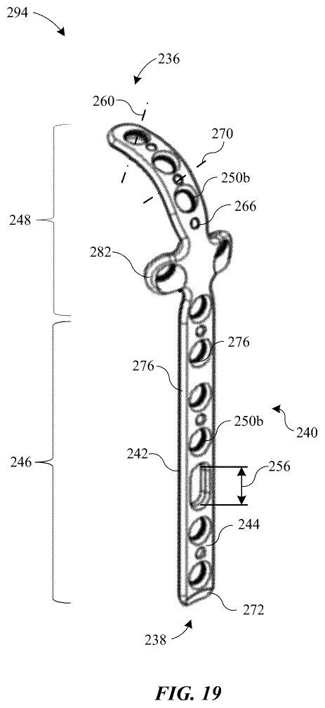

FIGS. 18A-19 show a fourth example of the proximal ulna plate 202. The fourth example of the proximal ulna plate 202 is similar to the very proximal ulna plate 232 except that the fourth example of the proximal ulna plate 202 does not extend as proximally or as distally along the olecranon 224 as the very proximal ulna plate 232. Accordingly, the fourth example of the proximal ulna plate 202 may be referred to a mini proximal ulna plate 294. The proximal portion 248 of the mini proximal ulna plate 294 may exclude the bulbus regions 252 and the triangular region 278 of the very proximal ulna plate 232. Furthermore, the mini proximal ulna plate 294 may be longitudinally symmetrical about the center line 254.

The mini proximal ulna plate 294 shown in FIGS. 18A-19 comprises twelve of the second openings 250b and does not include any of the first openings 250a. By excluding the first openings 250a, the mini proximal ulna plate 294 may have a lower profile (e.g., may be thinner) than the other implementations of the proximal ulna plate 202 described herein, which may mitigate post-operative soft tissue irritation. Seven of the second openings 250b may be spaced apart from each other longitudinally along the shaft portion 246 (e.g., along the center line 254), and three of the second openings 250b may be spaced apart from each other longitudinally along the proximal portion 248 (e.g., along the center line 254). Although the mini proximal ulna plate 294 shown in FIGS. 18A-19 comprises twelve of the openings 250, longer and shorter length implementations of the mini proximal ulna plate 294 having different quantities and configurations of the openings 250 are also contemplated by this disclosure.

The mini proximal ulna plate 294 may include two extension portions 282, one of which extends from the proximal portion 248 in a medial direction and one of which extends from the proximal portion 248 in a lateral direction to sit on respective posteromedial and posterolateral sides of the olecranon 224. Accordingly, the first surface 242 of the mini proximal ulna plate 294 along the extension portions 282 may include an anatomical contour that substantially matches the surface 210 of the respective posteromedial and posterolateral sides of the olecranon 224. Each of the extension portions 282 may include one of the second openings 250b.

Similar to the very proximal ulna plate 232, the mini proximal ulna plate 294 may be configured to support variable positioning of the fasteners 208 and dynamic compression of the ulna 206 (or portions thereof). Accordingly, in the implementation shown in FIGS. 18A-19, the third-most distal of the openings 250 may include an oblong profile. In other implementations, however, others of the openings 250 may include an oblong profile.

The openings 250 of the mini proximal ulna plate 294 may each define the fastener insertion axis 260 (e.g., the nominal fastener insertion axis) to target certain regions of the ulna 206 while avoiding other regions and surgical hardware. Accordingly, in some implementations, at least some of the openings 250 located on the shaft portion 246 may define the fastener insertion axis 260 to be substantially normal to the second surface 244 of the mini proximal ulna plate 294 such that the fasteners 208 have a substantially transverse trajectory into the ulna 206. The openings 250 located on the proximal portion 248 and the extension portions 282 may each define the fastener insertion axis 260 (e.g., the nominal fastener insertion axis) to target the coronoid 226, the olecranon 224, the intermedullary canal 222, and/or other regions of the proximal aspect 204 of the ulna 206. For example, as best shown in FIG. 18A, the first through third-most proximal of the openings 250 may each define the fastener insertion axis 260 to aim generally transversely and distally toward the coronoid 226 and/or the anterior side of the proximal metaphysis 218. The openings 250 located on the extension portions 282 may each define the fastener insertion axis 260 to aim transversely and proximally toward a proximal region of the olecranon 224.

Still referring to FIGS. 18A-19, to support provisional fixation of the medially extending proximal ulna plate 286 to the ulna 206, the mini proximal ulna plate 294 may include the apertures 266 (e.g., six of the apertures 266) configured to receive respective ones of the stabilizers 268. The mini proximal ulna plate 294 shown in FIGS. 18A-19 includes three of the apertures 266 along the shaft portion 246, in which one of the apertures 266 is each positioned between the first and second-most distal of the second openings 250b, between the fourth and fifth-most distal of the second openings 250b, and between the sixth and seventh-most distal of the second openings 250b. The mini proximal ulna plate 294 may also include three of the apertures 266 on the proximal portion 248, in which one of the apertures 266 is each positioned between the first and second-most proximal of the second openings 250b, between the second and third-most proximal of the second openings 250b, and between third-most proximal of the second openings 250b and the extension portions 282. In other implementations, however, the apertures 266 may be positioned along the less proximal ulna plate 234 according to some other configuration.

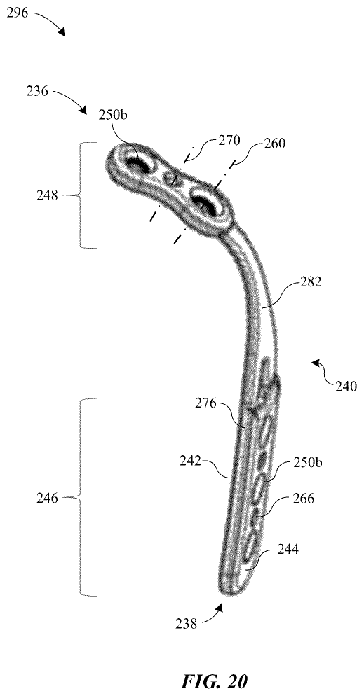

FIG. 20 shows a fifth example of the proximal ulna plate 202. The fifth example of the proximal ulna plate 202 is similar to the very proximal ulna plate 232 except that the fifth example of the proximal ulna plate 202 does not extend as proximally or as distally along the olecranon 224 as the very proximal ulna plate 232 and is primarily configured for re-establishment of the olecranon 224 after an olecranon osteotomy. Surgeons often perform an olecranon osteotomy to provide the surgeon a view of the distal humerus, in which the surgeon removes of a portion of the olecranon of the ulna 206 that would otherwise inhibit the surgeon's view of the distal humerus. However, after visualization of the distal humerus is no longer required (e.g., at the end of the procedure), the portion of the olecranon 224 that was removed must be reconnected to the ulna 206. A surgeon may use a band wire, a large screw, or other hardware to reconnect the removed portion to the olecranon 224. Such techniques, however, often result in soft tissue irritation and/or may require the future removal of the applicable hardware. Thus, an improved system for quickly repairing an olecranon osteotomy may be desired.

Accordingly, the fifth example of the proximal ulna plate 202 may be referred to an osteotomy proximal ulna plate 296. The proximal portion 248 of the mini proximal ulna plate 294 may exclude the bulbus regions 252 and the triangular region 278 of the very proximal ulna plate 232. Furthermore, the osteotomy proximal ulna plate 296 may be longitudinally symmetrical about the center line 254. In some implementations, the osteotomy proximal ulna plate 296 may include a band 288 that connects the shaft portion 246 to the proximal portion 248. The band 288 may have a thickness that is less than the shaft portion 246 and/or the proximal portion 248. Accordingly, the band 288 may be more malleable than the shaft portion 246 and/or the proximal portion 248. In such an implementation, the band 288 may enable the osteotomy proximal ulna plate 296 to be easily bent to form the osteotomy proximal ulna plate 296 to the proximal aspect 204 of the ulna 206. Furthermore, in some implementations the band 288 may be compliant (e.g., elastic) such that the osteotomy proximal ulna plate 296 may conform to the surface 210 of the olecranon 224 without plastic deformation.

The osteotomy proximal ulna plate 296 shown in FIG. 20 comprises five of the second openings 250b and does not include any of the first openings 250a. By excluding the first openings 250a, the osteotomy proximal ulna plate 296 may have a lower profile (e.g., may be thinner) than the other implementations of the proximal ulna plate 202 described herein, which may mitigate post-operative soft tissue irritation. Three of the second openings 250b may be spaced apart from each other longitudinally along the shaft portion 246 (e.g., along the center line 254), and two of the second openings 250b may be spaced apart from each other longitudinally along the proximal portion 248 (e.g., along the center line 254). Although the osteotomy proximal ulna plate 296 shown in FIG. 20 comprises five of the openings 250, longer and shorter length implementations of the osteotomy proximal ulna plate 296 having different quantities and configurations of the openings 250 are also contemplated by this disclosure.

The openings 250 of the osteotomy proximal ulna plate 296 may each define the fastener insertion axis 260 (e.g., the nominal fastener insertion axis) to target certain regions of the ulna 206 while avoiding other regions and surgical hardware. Accordingly, in some implementations, at least some of the openings 250 located on the shaft portion 246 may define the fastener insertion axis 260 to be substantially normal to the second surface 244 of the mini proximal ulna plate 294 such that the fasteners 208 have a substantially transverse trajectory into the ulna 206. The openings 250 located on the proximal portion 248 may each define the fastener insertion axis 260 (e.g., the nominal fastener insertion axis) to target the coronoid 226, the olecranon 224, the intermedullary canal 222, and/or other regions of the proximal aspect 204 of the ulna 206.

Still referring to FIG. 20, to support provisional fixation of the osteotomy proximal ulna plate 296 to the ulna 206, the osteotomy proximal ulna plate 296 may include the apertures 266 (e.g., three of the apertures 266) configured to receive respective ones of the stabilizers 268. The osteotomy proximal ulna plate 296 shown in FIG. 20 includes two of the apertures 266 along the shaft portion 246, in which one of the apertures 266 is positioned between each of the first and second-most distal of the second openings 250b, and between the second and third-most distal of the second openings 250b. The osteotomy proximal ulna plate 296 may also include one of the apertures 266 on the proximal portion 248 between the first and second-most proximal of the second openings 250b. In other implementations, however, the apertures 266 may be positioned along the less proximal ulna plate 234 according to some other configuration.

Although the invention has been described in detail with reference to specific embodiments, it will be apparent to one skilled in the art that various changes and modifications can be made without departing from the spirt and scope of the invention. Thus, it is intended that the invention covers the modifications and variations of this invention provided they come within the scope of the appended claims and their equivalents. It is expressly intended, for example, that all ranges broadly recited in this document include within their scope all narrower ranges which fall within the broader rangers. It is also intended that the components of the various devices disclosed above may be combined or modified in any suitable configuration.

What is claimed is:

1. A plate configured to support an ulna, the plate extending longitudinally to define a proximal end and a distal end opposite the proximal end, the plate comprising:
a first surface and a second surface opposite the first surface, wherein at least a portion of the first surface is configured to contact a surface of the ulna;
first openings spaced apart from one another longitudinally along a shaft portion of the plate that corresponds to at least one of a proximal metaphysis or a diaphysis of the ulna;
second openings spaced apart from one another along a proximal portion of the plate that corresponds to at least one of the proximal metaphysis or a proximal epiphysis of the ulna, wherein the first openings and the second openings each define a fastener insertion axis for a fastener to be inserted therethrough, wherein at least some of the second openings define the fastener insertion axis to be generally normal to the second surface of the plate, and wherein a diameter of each of the first openings is larger than a diameter of each of the second openings; and
apertures each adjacent to at least one of the first openings or the second openings, each of the apertures defining a stabilizer insertion axis for a stabilizer to be inserted therethrough, wherein the stabilizer insertion axis is generally parallel with the fastener insertion axis of the at least one of the first openings or the second openings to which each of the apertures is adjacent,
wherein the first surface of the plate is shaped to contour to a portion of the surface of the ulna that extends along an olecranon of the ulna.

2. The plate of claim 1, wherein at least some of the first openings and the second openings include locking features configured to prevent respective fasteners from loosening after being inserted therethrough.

3. The plate of claim 2, wherein the fastener insertion axis of each of the first openings and the second openings include a nominal fastener insertion axis and a conical range of fastener insertion axes surrounding the nominal fastener insertion axis, and wherein the stabilizer insertion axis is generally parallel with the nominal fastener insertion axis of the at least one of the first openings or the second openings to which each of the apertures is adjacent.

4. The plate of claim 3, wherein each of the first openings and the second openings defines the nominal fastener insertion axis to not intersect with the nominal fastener insertion axis of any other of the first openings and the second openings.

5. The plate of claim 4, wherein the second surface of the plate is beveled at the distal end of the plate.

6. The plate of claim 4, wherein at least one of the second openings defines the nominal fastener insertion axis to target a coronoid, a proximal region of the olecranon, or an intermedullary canal of the ulna from a posterior surface of the olecranon of the ulna.

7. The plate of claim 6, wherein a thickness of the proximal portion of the plate is tapered such that the proximal end of the plate is thinner than the shaft portion.

8. The plate of claim 7, wherein the proximal portion includes side that are arcuately curved to form one or more bulbus regions.

9. The plate of claim 8, wherein two of the second openings of the proximal portion are positioned laterally adjacent to one another at the proximal end of the plate, and wherein the two of the second openings each define the nominal fastener insertion axis to aim generally distally along a longitudinal axis of the ulna toward a diaphysis of the ulna.

10. The plate of claim 4, further comprising:
an extension plate that extends from the proximal portion of the plate in a medial direction, the extension plate including at least two of the second openings, wherein two of the second openings of the extension plate each define the nominal fastener insertion axis to target an intermedullary canal, a coronoid, or an olecranon of the ulna.

11. The plate of claim 10, further comprising:
recesses spaced apart from one another along sides of the shaft portion of the plate.

12. A plate configured to support an ulna, the plate extending longitudinally to define a proximal end and a distal end opposite the proximal end, the plate comprising:
a first surface and a second surface opposite the first surface, wherein at least a portion of the first surface is configured to contact a surface of the ulna;
openings spaced apart from one another along the plate, at least some of the openings defining a nominal fastener insertion axis and a conical range of fastener insertion axes surrounding the nominal fastener insertion axis for a fastener to be inserted therethrough; and
apertures each adjacent to at least one of the openings, each of the apertures defining a stabilizer insertion axis for a stabilizer to be inserted therethrough, wherein the stabilizer insertion axis is generally parallel with the nominal fastener insertion axis of the at least one of the openings to which each of the apertures is adjacent,
wherein the first surface of the plate is shaped to contour to a portion of the surface of the ulna that extends along an olecranon of the ulna.

13. The plate of claim 12, wherein at least one of the openings includes an oblong profile that has a length, and wherein the fastener is configured to be inserted therethrough at locations along the length.

14. The plate of claim 12, wherein the second surface of the plate is beveled at the distal end of the plate.

15. The plate of claim 12, wherein some of the openings define the nominal fastener insertion axis to be substantially normal to the second surface of the plate, and wherein some of the openings define the nominal fastener insertion axis to target a coronoid, a proximal region of the olecranon, or an intermedullary canal of the ulna.

16. The plate of claim 15, further comprising:
two extension portions, one of the extension portions extending medially from the plate and one of the extension portions extending laterally from the plate, wherein each of the extension portions includes one of the openings, and wherein the openings of each of the extension portions define the nominal fastener insertion axis to target a proximal region of the olecranon of the ulna.

17. The plate of claim 16, wherein the plate is symmetrical about a center line that longitudinally bisects the plate.

18. A plate configured to support an ulna, the plate extending longitudinally to define a proximal end and a distal end opposite the proximal end, the plate comprising:
a proximal portion located at the proximal end of the plate and a shaft portion located at the distal end of the plate, wherein each of the proximal portion and the shaft portion comprise:
a first surface and a second surface opposite the first surface, wherein at least a portion of the first surface is configured to contact a surface of the ulna;
at least one opening defining a fastener insertion axis for a fastener to be inserted therethrough; and
at least one aperture defining a stabilizer insertion axis for a stabilizer to be inserted therethrough; and
a band connecting the proximal portion to the shaft portion, wherein the band is curved to contour around a posterior surface of an olecranon of the ulna,
wherein the at least one opening of the proximal portion defines the fastener insertion axis to aim distally toward a diaphysis of the ulna, and
wherein the at least one opening of the shaft portion defines the fastener insertion axis to target a coronoid of the ulna.

19. The plate of claim 18, wherein the band has a thickness less than other thicknesses of the proximal portion of the plate and the shaft portion of the plate.

20. The plate of claim 18, wherein the plate is symmetrical about a center line that longitudinally bisects the plate.
